(19) United States
(12) Patent Application Publication
(10) Pub. No.: US 2016/0046722 A1
(43) Pub. Date: Feb. 18, 2016

(54) NOVEL MEDICAMENTS COMPRISING AN ANTIBODY COMPOSITION ENRICHED WITH PREDOMINANT CHARGE ISOFORM

(71) Applicant: LABORATOIRE FRANCAIS DU FRACTIONNEMENT ET DES BIOTECHNOLOGIES, Les Ulis (FR)

(72) Inventors: Guillaume Chevreux, Paris (FR); Nicolas Bihoreau, Orsay (FR)

(73) Assignee: LABORATOIRE FRANCAIS DU FRACTIONNEMENT ET DES BIOTECHNOLOGIES, Les Ulis (FR)

(21) Appl. No.: 14/776,725

(22) PCT Filed: Mar. 14, 2014
(86) PCT No.: PCT/EP2014/055179

§ 371 Date: Sep. 14, 2015

(30) Foreign Application Priority Data
Mar. 15, 2013 (FR) 1352360

(51) Int. Cl. C07K 16/28

(52) U.S. Cl. CPC C07K 16/2896; C07K 2317/24; C07K 2317/21

(57) ABSTRACT

The present invention lies in the technical field of antibody therapies involving a mechanism of target-cell destruction by ADCC. It relates to purified antibody compositions, obtained by chromatographic fractionation of the various charge isoforms naturally present in an antibody composition and combining one or more chromatographic fractions corresponding to the predominant peak of the chromatogram, the resulting monoclonal antibody composition being enriched in said predominant peak, said peak representing at least 85% of the chromatogram of the composition obtained, for use as a medicament.

### FIELD OF THE INVENTION

The present invention is located in the technical field of antibody therapies involving a mechanism of destructing target cells via ADCC. It relates to purified antibody compositions, obtained by fractionating by chromatography the different charge isoforms naturally present in an antibody composition and combining one or more chromatographic fractions corresponding to the majormajor peak of the chromatogram, the thereby obtained monoclonal antibody composition being enriched in said major majorpeak, the latter representing at least 85% of the chromatogram of the obtained composition, for a use as a medicament.

### PRIOR ART

During the last decade there has been a strong development of passive immunotherapy treatments by means of antibodies, often monoclonal antibodies, in various therapeutic fields: cancers, prevention of allo-immunization in Rhesus negative pregnant women, infectious diseases, inflammatory diseases and notably auto-immune diseases.

Although passive immunotherapy treatments by means of antibodies have today shown their therapeutic benefit, the observed clinical reaction levels are still insufficient, and therefore there is a need for more efficient antibody compositions, giving the possibility of increasing clinical responses and of administering smaller doses, in order to limit secondary effects.

Like any biological product, a composition of antibodies is by nature heterogeneous. Indeed, antibody compositions used in therapy are produced in biological systems (cells, transgenic animals or plants), in which proteins in general, and therefore antibodies in particular, are subject to a number of post-translational modifications (enzymatic modifications or degradations), which will vary from one antibody molecule to another and thereby generate micro-heterogeneity within the produced antibody composition.

Antibodies are glycoproteins consisting of four polypeptide chains: two generally identical heavy chains (so-called “H” chains for “heavy” chains) and two generally identical light chains (so-called “L” chains for “light” chains) associated with a variable number of disulfide bridges and non-covalent interactions. These chains form a Y-shaped structure, the heavy chain contributing to the stem of the Y and to half of each arm of the Y, the light chain contributing to half of each arm of the Y. Each light chain consists of a constant domain (CO and of a variable domain (V_{L}); the heavy chains consist of a variable fragment (V_{H}) and of 3 or 4 constant fragments (C_{H1} to C_{H3} or C_{H4}) depending on the isotype of the antibody (IgGs comprise 3 constant fragments C_{H1} to C_{H3}). The association of the light chain (V_{L}+C_{L}) and of the V_{H} and C_{H1} domains of the heavy chain forms fragment Fab, the associated domains VL and VH being responsible for the recognition of the antigen. Constant domains (C_{H2} and C_{H3}) or (C_{H2} to C_{H4}) of both heavy chains form constant Fc fragment.

Antibodies are known to be subjected to the following post-translational modifications: terminal modifications of heavy or light chains, glycosylation of the Fc portion (and optionally Fabs), deamidation, isomerization, oxidation, fragmentation, and aggregation (see Vlasak et al.—2008).

Most post-translational modifications lead to alteration of the surface charge properties of the antibody, either directly by modifying the number of charged groups, or indirectly by introducing structural modifications, which themselves modify the local distribution of the charged residues or change their pKa. All these modifications therefore also generate micro-heterogeneity, many isoforms with different charges of a same antibody, with distinct isoelectric points (pI), thus cohabiting within an antibody composition (see Vlasak et al.—2008).

Among post-translational modifications, glycosylation of the constant portion Fc of the antibodies is today well known for strongly influencing many biological properties of the antibody: half-life in vivo (see Wright et al.—1994), ability to induce an ADCC response (antibody-dependent cytotoxic cell response, see Satoh et al.—2006, Presta et al.—2006), a CDC response (complement-dependent cytotoxic response, see Wright et al.—1994, Presta et al.—2006), etc. In particular, the content of the antibody composition in fucosylated glycan forms is today known to very strongly affect the ability of the composition to induce an ADCC response in vivo. On the contrary, although many articles aim at characterizing the charge isoforms present in an antibody composition for justifying reproducibility and quality of the commercial batches of monoclonal antibodies, other post-translational modifications leading to the existence of many distinct charge isoforms of a same antibody within an antibody composition have up to now been considered as having little or no impact on the biological properties of antibodies in vivo. Thus, although it is generally considered as indispensable in the prior art to track the quality of commercial batches of antibodies as regards charge isoforms, this tracking is considered as pure tracking of the quality of the products and there has never been a proposal to use a purified fraction of an antibody composition, strongly enriched in a particular charge isoform, for a therapeutic purpose. Indeed, in the absence of demonstrating a significant effect on at least certain biological properties of the antibody composition, there was no reason not to use the entire composition, to complicate the preparation method and reduce the yield. Now, as indicated above, except for glycosylation, the other post-translational modifications leading to the existence of many distinct charge isoforms of an antibody within an antibody composition were up to now considered as not altering the biological properties of the antibodies.

One of the modifications leading to the occurrence of several charge isoforms is the enzymatic cleavage of C-terminal lysine in the heavy chains of the antibody. Such a cleavage occurs at different levels depending on the antibody molecules, as soon as the antibody is produced in a cell expressing a carboxypeptidase. The presence of a C-terminal lysine gives a rather basic nature, because of the side chain of lysine. Its cleavage on either or both heavy chains therefore generates more acidic isoforms. Generally, there are isoforms with 0, 1 or 2 C-terminal lysines on heavy chains, thus generating three isoforms with slightly different pIs (see Vlasak et al.—2008). On this particular modification, Antes et al.—2007 describe the analysis by isoelectric focusing (IEF) of batches of a humanized monoclonal anti-Lewis-Y IGN311 antibody used in passive immunotherapy of cancers produced in the presence or in the absence of serum. The authors show that the profiles of charge isoforms of antibody compositions produced in the presence or in the absence of serum are different, the composition produced in the absence of serum being less affected than that produced in the presence of serum by enzymatic cleavage of the C-terminal lysine of the heavy chain of the antibody. The analysis of the effect of this modification on the respective abilities of both compositions to induce a CDC response (via the complement) has not shown any significant effect related to this modification.

Another type of modification leading to the occurrence of several charge isoforms within an antibody composition is the cyclisation of N-terminal glutamine or glutamic acid residues, which leads to the formation of a pyroglutamate (pE) group and therefore to more acidic isoforms. This modification occurs systematically, at different levels, in the whole antibody composition, but is not considered as capable of affecting the functional properties of the antibody (see Vlasak et al.—2008). Still another type of modification leading to the occurrence of several charge isoforms within an antibody composition is the formation of covalent adducts and notably glycation phenomena (non-enzymatic addition of sugars), in particular on lysine residues, which generates more acidic isoforms. This type of modification is also considered as not being able to affect the functional properties of the antibody (see Vlasak et al.—2008).

Another usual type of modification leading to the occurrence of several charge isoforms within an antibody composition is deamidation of asparagine residues and the isomerization of aspartate residues, which generates more acidic isoforms. In the constant portion of the antibodies, the asparagine residues sensitive to deamidation phenomena are located in the CH3 domain, away from the binding sites to FcRn receptor and to FcγR receptors. These modifications are therefore generally considered as not being able to affect the functional properties of the antibody (see Vlasak et al.—2008).

Khawli et al.—2010 and Gandhi et al.—2011 describe the separation with chromatography techniques using a cations exchanging resin of major, acidic and basic isoforms of a monoclonal antibody composition used in passive immunotherapy; the analysis of post-translational modifications leading to the existence of several isoforms; as well as the study of the pharmacokinetic properties and of certain functional properties of three purified fractions (acidic, major and basic fractions). In both cases, the chromatogram of the native composition always shows a major peak, surrounded with peaks comprising acidic isoforms and peaks comprising basic isoforms. The identified post-translational modifications notably include the reduction of certain disulfide bridges (Khawli et al.—2010), glycations (Khawli et al.—2010; Gandhi et al.—2011), deamidations (Khawli et al.—2010; Gandhi et al.—2011), cleavage of C-terminal lysines of heavy chains (Khawli et al.—2010; Gandhi et al.—2011), the presence of aggregates (Gandhi et al.—2011), oxidation phenomena (Gandhi et al.—2011). The analysis of the pharmacokinetic properties (FcRn binding and test in vivo in Khawli et al.—2010) did not allow any demonstration of a significant difference in behavior at the three tested purified fractions. In both articles, the capability of the three purified fractions of inhibiting in vitro the proliferation of a cell line expressing the antigen for which the antibody is specific, in the absence of effector cells, was also tested. Such a test gives the possibility to demonstrate the ability to bind to the antigen and to induce apoptosis. Although the acidic fraction in both articles had a very slightly lower capability, the results are not significant and no significant difference was therefore observed between the three purified fractions. Further, the fraction enriched in major isoform did not have enhanced abilities as compared with the total antibody composition, before separation of the three fractions.

Moreover, other documents describe how to analyze and/or separate certain charge isoforms of antibodies, but without comparing the effector properties of the different isoforms. Thus, EP1308456 and WO2004/024866 describe chromatography methods aiming at removing the acidic variants of a monoclonal antibody composition, without having tested the effector properties of the composition before and after purification. Also, WO2011/009623 describes a chromatography method aiming at suppressing the acidic variants or the basic variants of a monoclonal antibody composition, without having tested the effector properties of the composition before and after purification. Further, the method described in this document only allows suppression of a single type of variant and only the removal of acidic variants is actually applied.

Thus, except for glycosylation, which is known for having effects on the functional properties of the antibodies, the elements available in the prior art concerning the other post-translational modifications generating several charge isoforms (from different modifications brought to the major isoform), suggest that these modifications do not have any impact on the functional properties of the antibodies. However, the inventors have surprisingly found that a fraction purified by chromatography, enriched in the major charge isoform of an antibody composition, has a significantly greater ability to induce an effector response via CD16 receptor by the effector cells expressing this receptor. Thus, a purified fraction enriched in the major charge isoform of an antibody composition gives the possibility of inducing a stronger ADCC response and a stronger CDC response in vivo, and therefore of increasing the clinical responses and/or reducing the administered doses, thereby limiting the secondary effects.

### SUMMARY OF THE INVENTION

The present invention therefore relates to a monoclonal antibody composition which may be obtained by a method comprising:
a) producing a monoclonal antibody composition from a cell clone, from a non-human transgenic animal or from a transgenic plant,
b) fractionating the composition obtained in step a) by chromatography, and
c) combining one or several chromatography fractions obtained in step b), corresponding to the major peak of the chromatogram, the thereby obtained monoclonal antibody composition being enriched in said major peak, the latter representing at least 85%, advantageously at least 86%, at least 87%, at least 88%, at least 89%, more advantageously at least 90%, at least 91%, at least 92%, at least 93%, at least 94%, or even at least 95%, at least 96%, at least 97%, at least 98%, at least 98.5%, at least 99%, or at least 99.5% of the chromatogram of the composition obtained in step c),
for its use as a medicament.

Advantageously, step b) is achieved by fractionating the composition obtained in step a) by standard ion exchange chromatography, by chromatofocusing, or by hydrophobic interactions chromatography .

Advantageously, ion exchange chromatography uses one of the following elution means:
an ionic force gradient; and/or
a pH gradient; or
a displacement molecule.

Advantageously, in such a composition for use as a medicament, at least 95%, advantageously at least 96%, at least 97%, at least 98%, or even at least 98.5%, at least 99%, or at least 99.5% of the heavy chains of the antibodies present in the composition do not comprise any C-terminal lysine residue.

The invention also relates to a monoclonal antibody composition, wherein at least 95%, advantageously at least 96%, at least 97%, at least 98%, or even at least 98.5%, at least 99%, or at least 99.5% of the heavy chains of the antibodies present in the composition do not comprise any C-terminal lysine residue, for its use as a medicament.

In the compositions for use as a medicament according to the invention, the antibody is advantageously directed against a non-ubiquitous antigen present on healthy donor cells, an antigen of a cancer cell, or an antigen of a cell infected by a pathogenic agent.

In particular, the following embodiments are preferred:
the antibody is an anti-Rhesus (D) antibody and the composition is intended for preventing allo-immunization in Rhesus-negative individuals.
the antibody is directed against an antigen of a cancer cell and the composition is intended for treating a cancer,
the antibody is directed against an antigen of a cell infected by a pathogenic agent and the composition is intended for treating an infection by said pathogenic organism,
the antibody is directed against an antigen of an immune cell and the composition is intended for treating an autoimmune disease.

In an advantageous embodiment, in a composition for use as a medicament according to the invention, the antibody comprises a modification of the Fc fragment increasing its binding to FcγRIII receptor and its effector properties via FcγRIII receptor. The composition for use as a medicament according to the invention may notably comprise mutations in the Fc fragment increasing its binding to FcγRIII receptor and/or a low fucose content. In particular, advantageously, the antibodies present in the composition have on their N-glycosylation sites of the Fc fragment glycan structures of the biantennary type, with a fucose content of less than 65%.

In an advantageous embodiment, in a composition for use as a medicament according to the invention, the antibody comprises a modification of the Fc fragment increasing its binding to the protein C1q and its effector properties via the complement.

The present invention also relates to the use of a chromatography fractionation step for increasing the ability of a monoclonal antibody composition directed against a given antibody to induce cell cytotoxicity depending on the antibody (ADCC) of target cells expressing said antigen by effector cells of the immune system expressing FcγRIII receptor (CD16).

The present invention also relates to the use of a chromatography fractionation step for increasing the ability of a monoclonal antibody composition directed against a given antibody to induce complement-dependent cytotoxicity (CDC) of target cells expressing said antigen by the complement.

### DESCRIPTION OF THE FIGURES

FIG. 1. Chromatograms obtained for three separations by chromatofocusing of an anti-CD20 antibody composition (anion exchange resin (column Mono™ P marketed by GE Life Sciences) with elution by a decreasing pH gradient (from 9.5 to 8.0 by using two buffers: buffer A (diethanolamine 25 mM), buffer B (polybuffer 96+pharmalyte 8-10.5)). The antibody composition was desalted, and 20 mg were injected onto the column. 2 mL fractions were collected. The fractions 33 to 50 were collected for analysis.

FIG. 2. Superposition of 11 chromatograms corresponding to eleven separations by cation exchange chromatography (same column and elution as A). The fractions F1 to F20 were collected and grouped per peaks: P1 (acid, F1 to F3), P2 (acid, F4 and F5), P3 (acid, F6), P4 (main peak, F7 to F10), P5 (basic, F11), P6 (basic, F12 to F14), P7 (basic, F15 to F17), and P8 (basic, F18 to F20).

FIG. 3. Chromatograms of the anti-CD20 antibody composition purified by CEX. A. Chromatogram of the anti-CD20 antibody composition before purification. B. Chromatogram of the composition formed by assembling fractions 1 to 20 corresponding to the major peak of the chromatogram before separation (A). The percentage of the various peaks is indicated.

FIG. 4. Binding to CD16 (Biacore) of fractions purified by cation exchange chromatography. The binding to CD16 of each sample is expressed as a percentage of the binding to CD16 of a reference sample.

FIG. 5. CD16 activity of fractions purified by chromatofocusing (A) or by cation exchange chromatography (B). The CD16 activity (secretion of IL-2 by CD16 Jurkat cells) of each sample is expressed as a percentage of the CD16 activity of a reference sample.

FIG. 6. Complement-dependent cytotoxicity (CDC) of fractions purified by cation exchange chromatography. The CDC response of each sample is expressed as a percentage of the CDC response of a reference sample.

### DETAILED DESCRIPTION OF THE INVENTION

The present invention therefore relates to a monoclonal antibody composition which may be obtained by a method comprising:
a) producing a monoclonal antibody composition from a cell clone, from a non-human transgenic animal or from a transgenic plant,
b) fractionating the composition obtained in step a) by chromatography, and
c) combining one or several chromatographic fractions obtained in step b), corresponding to the major peak of the chromatogram, the thereby obtained monoclonal antibody composition being enriched in said major peak, the latter representing at least 85%, advantageously at least 86%, at least 87%, at least 88%, at least 89%, more advantageously at least 90%, at least 91%, at least 92%, at least 93%, at least 94%, or even at least 95%, at least 96%, at least 97%, at least 98%, at least 98.5%, at least 99%, or at least 99.5% of the chromatogram of the composition obtained in step c),
for its use as a medicament.

In step a), a monoclonal antibody composition is produced from a cell clone, from a transgenic animal or from a transgenic plant.

By “antibody” or “immunoglobulin”, is meant a molecule comprising at least one domain for binding to a given antigen and a constant domain comprising an Fc fragment capable of binding to FcR receptors. In most mammals, like humans and mice, an antibody consists of 4 polypeptide chains: 2 heavy chains and 2 light chains connected together through a variable number of disulfide bridges ensuring flexibility to the molecule. Each light chain consists of a constant domain (CL) and of a variable domain (VL); the heavy chains consists of a variable domain (VH) and of 3 or 4 constant domains (CH1 to CH3 or CH1 to CH4) according to the isotype of the antibody. In a few rare mammals, like camels and lamas, the antibodies only consist of two heavy chains, each heavy chain comprising a variable domain (VH)

Variable domains are involved in recognition of the antigen, while constant domains are involved in biological, pharmacokinetic and effector properties of the antibody. Unlike variable domains, for which the sequence strongly varies from one antibody to another, constant domains are characterized by an amino acid sequence very close from one antibody to the other, typical of the species and of the isotype, with optionally a few somatic mutations. The Fc fragment naturally consists of the constant region of the heavy chain excluding domain CH1, i.e. of the lower boundary region and of the constant domains CH2 and CH3 or CH2 to CH4 (depending on the isotype). In human IgG1, the complete Fc fragment consists of the C-terminal portion of the heavy chain starting from the cysteine residue in position 226 (C226), the numbering of amino acid residues in the Fc fragment being in all the present description that of the index EU described in Edelman et al.—1969 and Kabat et al.—1991. The corresponding Fc fragments of other types of immunoglobulins may easily be identified by one skilled in the art by alignments of sequences.

The Fc fragment is glycosylated in the CH2 domain with the presence, on each of the 2 heavy chains, of an N-glycan bound to the asparagine residue in position 297 (Asn 297).

The following binding domains, located in Fc, are important for the biological properties of the antibody:
domain for binding to FcRn receptor, involved in the pharmacokinetic properties (half-life in vivo) of the antibody: Different data suggests that certain residues located at the interface of the CH2 and CH3 domains are involved in the binding to FcRn receptor.
domain for binding to the protein of the complement C1q, involved in the CDC response (for “complement-dependent cytotoxicity”): located in the CH2 domain;
domain for binding to FcR receptors, involved in the responses of the phagocytosis or ADCC (for “antibody-dependent cell cytotoxicity”) type: located in the CH2 domain.

In the sense of the invention, the Fc fragment of an antibody may be natural, as defined above, or else may have been modified in various ways, provided that it comprises a functional domain for binding to FcR receptors (FcγR receptors for IgGs), and preferably a functional domain for binding to receptor FcRn. The modifications may include the deletion of certain portions of the Fc fragment, provided that the latter contains a functional domain for binding to receptors FcR (receptors FcγR for IgGs), and preferably a functional domain for binding to receptor FcRn. The modifications may also include various substitutions of amino acids able to affect the biological properties of the antibody, provided that the latter contains a functional domain for binding to receptors FcR, and preferably a functional domain for binding to receptor FcRn. In particular, when the antibody is an IgG, it may comprise mutations intended to enhance the binding to receptor FcγRIII (CD16), as described in WO00/42072, Shields et al.—2001, Lazar et al.—2006, WO2004/029207, WO/2004063351, WO2004/074455. Mutations permitting to enhance the binding to receptor FcRn and therefore the half-life in vivo may also be present, as described for example in Shields et al.—2001, Dall'Acqua et al.—2002, Hinton et al.—2004, Dall'Acqua et al.—2006(a), WO00/42072, WO02/060919A2, WO2010/045193, or WO2010/106180A2. Other mutations, such as those permitting to reduce or increase the binding to the proteins of the complement and therefore the CDC response, may be present or not (see WO99/51642, WO2004074455A2, Idusogie et al.—2001, Dall'Acqua et al.—2006(b), and Moore et al.—2010).

By “monoclonal antibody” or “monoclonal antibody composition”, is meant a composition comprising antibody molecules having an identical and unique antigen specificity. The antibody molecules present in the composition may vary as regards their post-translational modifications, and notably as regards their glycosylation structures or their isoelectric point, but have all been encoded by the same heavy and light chain sequences and therefore have, before any post-translational modification, the same protein sequence. Certain differences in protein sequences, related to post-translational modifications (such as for example the cleavage of the C-terminal lysine of the heavy chain, deamidation of asparagine residues and/or isomerization of aspartate residues), may nevertheless exist between the various antibody molecules present in the composition.

The monoclonal antibody present in the composition used as a medicament within the scope of the invention may advantageously be chimeric, humanized, or human. Indeed, this gives the possibility of avoiding immune reactions of the patient against the administered antibody.

By “chimeric” antibody, it is meant to designate an antibody which contains a natural variable region (light chain and heavy chain) derived from an antibody of a given species associated with constant regions of light chain and heavy chain of an antibody of a species heterologous to said given species. Advantageously, if the monoclonal antibody composition for its use as a medicament according to the invention comprises a chimeric monoclonal antibody, the latter comprises human constant regions. Starting from a non-human antibody, a chimeric antibody may be prepared by using genetic recombinant techniques well known to one skilled in the art. For example, the chimeric antibody may be produced by cloning for the heavy chain and the light chain a recombinant DNA including a promoter and a sequence coding for the variable region of the non-human antibody, and a sequence coding for the constant region of a human antibody. As for the methods for preparing chimeric antibodies, reference may for example be made to document Verhoeyn et al.—1988.

By “humanized” antibody, it is meant to designate an antibody which contains CDR regions derived from an antibody of non-human origin, the other portions of the antibody molecule being derived from one (or from several) human antibodies. Further, certain of the residues of the backbone segments (called FR) may be modified for retaining the binding affinity (Jones et al.—1986; Verhoeyen et al.—1988; Riechmann et al.—1988). The humanized antibodies according to the invention may be prepared by techniques known to one skilled in the art such as “CDR grafting”, “resurfacing”, SuperHumanization, “Human string content”, “FR libraries”, “Guided selection”, “FR shuffling” and “Humaneering” techniques, as summarized in the review of Almagro et al.—2008.

By “human” antibody, is meant an antibody for which the whole sequence is of human origin, i.e. for which the coding sequences have been produced by recombination of human genes coding for antibodies. Indeed, it is now possible to produce transgenic animals (for ex. mice) which are capable, upon immunization, of producing a complete list of human antibodies in the absence of endogenous immunoglobulin production (see Jakobovits et al.—1993(a) and (b); Bruggermann et al.—1993; and Duchosal et al.—1992, U.S. Pat. Nos. 5,591,669, 5,598,369, 5,545,806, 5,545,807, 6,150,584). The human antibodies may also be obtained from phage display banks (Hoogenboom et al.—1991; Marks et al.—1991; Vaughan et al.—1996). The antibodies may be of several isotypes, depending on the nature of their constant region: constant regions γ, α, μ, ε and δ respectively correspond to IgG, IgA, IgM, IgE and IgD immunoglobulins. Advantageously, the monoclonal antibody present in a composition used as a medicament within the scope of the invention is of an IgG isotype. Indeed, this isotype shows an ability to generate ADCC (“Antibody-Dependent Cellular Cytotoxicity”) activity in the largest number of individuals (humans). γ constant regions comprise several sub-types: γ1, γ2, γ3, these three types of constant regions having the particularity of binding the human complement, and γ4, thereby generating sub-isotypes IgG1, IgG2, IgG3, and IgG4. Advantageously, the monoclonal antibody present in a composition used as a medicament within the scope of the invention is of an isotype IgG1 or IgG3, preferably IgG1.

The composition of monoclonal antibody may be produced by a cell clone, a non-human transgenic animal or a transgenic plant, by technologies well known to one skilled in the art.

Notably, cell clones producing the composition may be obtained by 3 main technologies:
1) Obtaining a hybridoma by fusion of a lymphocyte B producing the antibody of interest with an immortalized line,
2) Immortalizing a lymphocyte B producing the antibody of interest by the Epstein-Barr virus (EBV),
3) Isolating sequences coding for an antibody of interest (generally from a hybridoma or an immortalized lymphocyte B), cloning in one or several vector(s) expressing sequences coding for the heavy and light chains of the antibody, transforming a cell line by the expression vector(s) and separating the different obtained cell clones. An expression vector of heavy and light chains of the antibody comprises the elements required for expressing sequences encoding the heavy and light chains of the antibody, and notably a promoter, a codon for initiating the transcription, termination sequences, and suitable sequences for regulating transcription. These elements vary according to the host used for expression and are easily selected by one skilled in the art considering his/her general knowledge. The vector may notably be a plasmid or a virus. The transformation techniques are also well known to one skilled in the art.

Transformation of cell lines by one or several expression vectors of the sequences encoding the heavy and light chains of the antibody are most commonly used, in particular for obtaining chimeric or humanized antibodies.

The transformed cell line is preferably of eukaryotic origin and may notably be selected from insect, plant, yeast or mammal cells. The antibody composition may then be produced by cultivating the host cell under suitable conditions. Suitable cell lines for producing antibodies notably include cell lines selected from: SP2/0; YB2/0; IR983F; human myeloma Namalwa; PERC6; CHO lines, notably CHO-K-1, CHO-Lec10, CHO-Lec1, CHO-Lec13, CHO Pro-5, CHO dhfr-, or a CHO line deleted for the two alleles encoding gene FUT8 and/or gene GMD; Wil-2; Jurkat; Vero; Molt-4; COS-7; 293-HEK; BHK; K6H6; NSO; SP2/0-Ag 14, P3X63Ag8.653, duck embryo cell line EB66® (Vivalis); and rat hepatoma lines H4-II-E (DSM ACC3129), H4-II-Es (DSM ACC3130) (see WO2012/041768). In a preferred embodiment, the antibody is produced in one of the following lines: YB2/0; a CHO line deleted for the two alleles encoding gene FUT8 and/or gene GMD; embryo duck cell line EB66® (Vivalis); and rat hepatoma lines H4-II-E (DSM ACC3129), H4-II-Es (DSM ACC3130). In a preferred embodiment, the antibody is produced in YB2/0 (ATCC CRL-1662).

Alternatively, the antibody composition may be produced in a non-human transgenic animal.

A non-human transgenic animal may be obtained by directly injecting the gene(s) of interest (here, the rearranged genes coding for the heavy and light chains of the antibody) in a fertilized egg (Gordon et al.—1980). A non-human transgenic animal may also be obtained by introducing the gene(s) of interest (here, the rearranged genes coding for the heavy and light chains of the antibody) in an embryo stem cell and preparing the animal by a chimera aggregation method or a chimera injection method (see Manipulating the Mouse Embryo, A Laboratory Manual, Second edition, Cold Spring Harbor Laboratory Press (1994); Gene Targeting, A Practical Approach, IRL Press at Oxford University Press (1993)). A non-human transgenic animal may also be obtained by a cloning technique in which a nucleus, into which the gene(s) of interest (here, the rearranged genes coding of the heavy and light chains of the antibody) has(have) been introduced, is transplanted into an enucleated egg (Ryan et al.—1997; Cibelli et al.—1998, WO0026357A2). A non- human transgenic animal producing an antibody of interest may be prepared by the methods above. The antibody may then be accumulated in the transgenic animal and harvested, notably from the milk or the eggs of the animal. For producing antibodies in the milk of non-human transgenic animals, preparation methods are notably described in WO9004036A1, WO9517085A1, WO0126455A1, WO2004050847A2, WO2005033281A2, WO2007048077A2. Methods for purifying proteins of interest from milk are also known (see WO0126455A1, WO2007106078A2). The non-human transgenic animals of interest notably include mice, rabbits, rats, goats, bovines (notably cows), and poultry (notably chicken).

The antibody composition may be produced in a transgenic plant. Many antibodies have already been produced in transgenic plants and the technologies required for obtaining a transgenic plant expressing an antibody of interest and for recovering the antibody are well known to one skilled in the art (see Stoger et al.—2002, Fisher et al.—2003, Ma et al.—2003, Schillberg et al.—2005). It is also possible to influence the glycosylation obtained in the plants in order to obtain glycosylation close to that of natural human antibodies (without xylose) and with further slight fucosylation, for example by means of small interfering RNAs (Forthal et al.—2010).

In step b) of the method permitting to obtain a monoclonal antibody composition for use as a medicament according to the invention, the different charge isoforms of antibodies present in the composition obtained in step a) are separated by fractionating the composition obtained in step a) by chromatography. As explained in the introduction, any monoclonal antibody composition produced by a cell clone, a non-human transgenic animal or a transgenic plant is characterized by the presence of a certain number of charge isoforms or variants of a same monoclonal antibody. The presence of these different charge isoforms or variants is related to the existence of post-translational modifications leading to an alteration of the surface charge properties of the antibody, either directly by modifying the number of charge groups, or indirectly by introducing structural modifications, which themselves modify the local distribution of the charged residues or change their pKa. Each charge isoform or variant is characterized by its isoelectric point (pl, further called isoelectric hydrogen potential (pHI)), which corresponds to the pH (hydrogen potential) for which the global charge of this molecule is zero or, in other words, the pH for which the molecule is electrically neutral (zwitterionic form or mixed ion). At a given pH, the different charge isoforms or variants of a monoclonal antibody will therefore have variable net charges, those for which the pI is less than the pH bearing a negative charge (the molecule tends to yield its protons to the basic medium), those for which the pI is equal to the pH being neutral, and those for which the pI is greater than the pH bearing a positive charge (the molecule tends to retain its protons or capture some of them from the acidic medium). The different charge isoforms or variants of a monoclonal antibody are present in variable proportions, depending on the frequency of the post-translational modifications present on each variant. A monoclonal antibody composition generally comprises a major variant or isoform, accompanied by a plurality of so-called acidic or basic variants or isoforms, depending on whether their pI is less than or greater than that of the major isoform. Depending on the antibody, its mode of production and the purification steps which it may have already been subjected to, the proportions of acidic isoforms, of the major peak and of the basic isoforms (calculated from the chromatogram of an ion exchange chromatography), generally varies around the following values: 10 to 30% of acidic isoforms, 50 to 75% of major peak, and 8 to 20% of basic isoforms (see Farnan et al.—2009, Rea et al.—2011, Rea et al.—2012, Khawli et al.—2010, Zhang et al.—2011, WO2011/009623, and EP1308456). Because of their differences in terms of pI and of net charge at a given pH, the charge isoforms of antibodies present in a given antibody composition may be separated by different chromatographic technologies.

Chromatography is a technique for separating chemical substances (liquid or gas homogenous mixture) which is based on the behaviour differences between a running mobile phase and a stationary phase (or fixed phase). Chromatographic methods may be classified according to the nature of the phases used or to that of the phenomena applied in the separation.

In an embodiment of the invention, the fractionation of step b) is achieved by means of ion exchange chromatography. Indeed this allows separation of the charge isoforms of a same protein. In ion (anions or cations) exchange chromatography, the parameter which will allow the separation of the different constituents is their net charge.

The antibody composition is first loaded on an ion exchange resin. For this, positively (anion exchange chromatography) or negatively (cation exchange chromatography) charged resins (fixed or stationary phase) are used. The molecules with a charge opposite to that of the ions of the resin will be retained/fixed on the resin.

Any type of cation or anion exchange resin either strong or weak, known to one skilled in the art and suitable for separation of the antibody composition of interest may be used. Depending on its protein sequence, the average isoelectric point (pI) of an antibody composition generally varies between 5 and 9, most often between 7 and 9. For a pI of more than 8, a cation exchange resin is used. Conversely, for a pI of less than 6, an anion exchange resin is used. For a pI comprised between 6 and 8, both types of ion (cation or anion) exchange resins may be tested. Thus, even if a cation exchange chromatography (negatively charged resin) followed by elution with an ionic force gradient is most often used, it is also possible in certain cases to use an anion exchange chromatography (positively charged resin). The ion exchange resins generally consist of a cross-linked polymer or a gel, on which are grafted positively charged groups (anion exchange resin) or negatively charged groups (cation exchange resin). The cross-linked polymer or gel may notably be selected from dextran (eg: Sephadex®), agarose (eg: Sepharose®), cellulose, methacrylate polymers (eg: Fratogel®), vinyl polymers (eg: Fractoprep®) such as poly(styrene divinylbenzene) (eg: Monobeads™; Source™; Bio Mab NP-5 or NP-10; Sepax Antibodix™ NP1.7, NP3, NP5 and NP10). The gel may advantageously appear as beads, with an average diameter comprised between 10 and 200 μm.

For cation exchange resins, negatively charged groups are grafted on the cross- linked polymer, such as groups of the sulfopropyl (SP), methyl sulfonate (S) or carboxymethyl (CM) type.

For anion exchange resins, positively charged groups are grafted on the cross- linked polymer, such as groups of the quaternary ammonium type (Q), notably quaternary aminoethyl (QAE), diethylaminoethyl (DEAE), dimethylaminoethyl (DMAE), trimethylaminoethyl (TMAE), or dimethylaminopropyl (ANX).

Cation exchange resins which may be used within the scope of the present invention include the resins Source™ 15S or 30S, Mono-S (marketed by GE Life Sciences); ProPac® WCX (in particular ProPac® WCX—10), ProPac® SCX (in particular ProPac® SCX—10 or SCX—20), ProSwift WCX, MAbPac® SCX (in particular MAbPac® SCX—10) (marketed by Dionex); Bio Mab (in particular Bio Mab NP—5 or NP—10, marketed by Agilent), PL-SCX (marketed by Agilent); Sepax Antibodix™ (in particular Sepax Antibodix™ NP1.7, NP3, NP5 and NP10) (marketed by Sepax) (see Farnan et al.—2009, Khawli et al.—2010, Gandhi et al.—2011, Zhang et al.—2011, Rea et al.—2011 and McAtee et al.—2012). Also, anion exchange resins which may be used within the scope of the present invention include the resins Source™ 15Q or 30Q, Mono™-Q (marketed by GE Life Sciences); ProPac® WAX (in particular ProPac® WAX-10), ProPac® SAX (in particular ProPac® SAX—10) (marketed by Dionex).

Once the antibody composition is loaded on the ion exchange resin, different elution methods may be used for separating the charge isoforms.

The elution of the fixed molecules may notably be achieved by using an elution buffer (mobile phase) containing ions with a charge opposite to that of the ions of the resin, which will enter into competition with the fixed molecules for interacting with the charges borne by the resin. It is either possible to directly use a buffer containing a strong ion concentration (in order to elute all the molecules in one go) or on the contrary to gradually increase the ion concentration (this is then referred to as an ionic force gradient), which gives the possibility of successively detaching the different molecules depending on the force of their electrostatic interactions with the resin. Practically, in this last scenario, two buffer solutions are used, one of a low ion concentration and the other of a strong ion concentration. Two driven pumps suck up and mix both of these solutions according to a ratio which varies overtime (the proportion of the strong ion concentration solution gradually increasing). The product of this mixing is used in the column. Examples of specific methods for separating charge isoforms of an antibody composition with this technology are described in Gandhi et al.—2011. Rea et al.—2012 also described the principle of this technology, as well as how to suitably select the column, the buffers and the operating parameters for separating charge isoforms or variants of antibodies (see section 7 pages 447-451).

In an alternative ion exchange chromatography, the elution is achieved not with an ionic force gradient, but with a pH gradient. Indeed, many ionizable groups are pH sensitive . With an increasing pH gradient (i.e. by increasing the pH), the ionization of acid groups (negatively charged) is favored and the ionization of basic groups (positively charged) is unfavoured. By increasing the pH, the occurrence of a net negative charge is therefore favored for molecules bearing pH sensitive ionizable groups. An increasing pH gradient therefore also allows separation of the charge isoforms of an antibody composition fixed on a negatively charged resin (cation exchanger). With a decreasing pH gradient (i.e. by decreasing the pH), the ionization of basic groups (positively charged) is favored and the ionization of acid groups (negatively charged) is unfavoured. By decreasing the pH, the occurrence of a net positive charge for the molecules bearing pH sensitive ionizable groups is favored. A decreasing pH gradient therefore also gives the possibility of separating the charge isoforms of an antibody composition fixed on a positively charged resin (anion exchanger). Examples of specific methods for separating charge isoforms of antibodies by ion exchange chromatography with elution by a pH gradient are described in Farnan et al.—2009 and Rea et al.—2011. Rea et al.—2012 also described the principle of this technology, as well as how to suitably select the column, the buffers, and the operating parameters for separating charge isoforms or variants of antibodies (see section 8 pages 451-452). Example 1 also describes the separation of charge isoforms of an antibody composition by cation exchange chromatography and elution with an increasing pH gradient. In another alternative of ion exchange chromatography, the elution may also be achieved by combining an ionic force gradient and a pH gradient (a so-called “hybrid” elution), as described in Rea et al.—2012 (see section 9 page 453).

In still another alternative of ion exchange chromatography, called here “displacement ion exchange chromatography” and which also allows separation of the charge isoforms of an antibody composition, an ion (anion or cation) exchanger resin is also used as a fixed or stationary phase, but the elution is achieved not by an ionic force and/or pH gradient, but by means of a displacement molecule, i.e. a molecule having a strong affinity for the chromatography resin, which will come into competition for binding onto the resin with the antibody molecules fixed beforehand on the resin, and thus displace the antibody molecules having a lower affinity for the resin than the displacement molecule. The antibody molecules will thus be forced to migrate along the column by a displacement molecule wave. As the latter crosses the column, a new equilibrium is set up, wherein the antibody molecules come into competition with each other for the binding sites to the resin which remain available. During this dynamic balancing process, the different charge variants or isoforms of antibodies are separated according to their more or less affinity for the ion exchange resin. The principle of this chromatographic separation method, as well as of the resins, buffers and materials required for its application in order to separate the charge isoforms of an antibody composition are notably described in Khawli et al.—2010, Zhang et al.—2011, and McAtee et al.—2012.

In these different elution modes of ion exchange chromatography, any suitable elution (pH or ionic force gradient) or displacement buffer may be used, depending on the selected column. Examples of resins and associated buffers are described in Farnan et al.—2009, Khawli et al.—2010, Gandhi et al.—2011, Zhang et al.—2011, Rea et al.—2011 and McAtee et al.—2012.

Another chromatography technique which allows separation of the charge isoforms of an antibody composition is chromatofocusing. In this technique, the proteins are separated according to their isoelectric point (pI). This technique is based on the use of the association of a particular resin (fixed or stationary phase) and of a particular amphoteric buffer. Notably, obtaining a linear pH gradient requires an equal buffer capacity over the whole range of pH used for separation, hence the requirement of buffers specifically designed for this application and of resins substituted with charged buffer amines.

The principle of the separation is the following: a chromatofocusing resin is balanced with an initial buffer at a pH slightly greater than the highest required pH. An elution buffer (adjusted to the lowest required pH) is passed through the column and begins to titrate the amines of the resin and of the proteins. Gradually as the elution buffer passes through the column, the pH is reduced and a downward moving pH gradient is generated. The sample is applied to the column after having passed a first volume of elution buffers on the column. The proteins of the sample are titrated (adjustment of the pH) as soon as they are introduced into the column. Those which are at a pH above their pI are negatively charged and retained close to the top of the column (by binding to the positively charged amine groups). The proteins which are at a pH below their pI begin to migrate along the column with the buffer flow and will not bind to the column before attaining an area where the pH is greater than their pI. This is the beginning of the separation process.

Gradually, as the pH continues to decrease at the top of the column (time-dependent change of the pH gradient), any protein for which the pI is greater than the new pH will become positively charged, be repelled by the positively charged amine groups and begin to migrate along the column with the elution buffer, its migration being more rapid than that of the pH gradient. Gradually as this protein migrates along the column, the pH increases. When the protein attains an area where the pH is greater than its pI, it again becomes negatively charged and again binds to the column. It remains bound until the mobile pH gradient reduces the local pH below its pI, a moment when it again becomes positively charged and again begins to migrate. This process is repeated until the protein is eluted from the column at a pH close to its pI.

The name of this technology comes from a focusing effect of the technique. Indeed, in a pH lowering gradient, a protein may exist in three charge states: positive, negative or neutral. Further, in chromatofocusing, the state of charge of a protein varies continuously gradually as the pH gradient develops and as the protein migrates through the different pH areas of the column. The molecules at the rear of an area will more rapidly migrate than those at the front of this same area, gradually forming increasingly narrow bands of proteins, each band corresponding to one or several proteins with the same pI.

Thus, in chromatofocusing, the proteins having different pIs migrate at different rates through the column gradually as the pH gradient develops, continually binding and dissociating from the resin bearing positively charged buffer amine groups, while being gradually focused into narrow bands and finally eluted. The proteins with the highest pI are eluted first, while the protein with the lowest pI will be eluted last. The resin used for separation by chromatofocusing is based on a standard resin (cross-linked polymer or gel as described above, preferably as beads as described above), notably of the poly(styrene divinylbenzene) or cross-linked agarose type, the latter being characterized by the grafting of positively charged buffer amine groups. These positively charged buffer amine groups are notably secondary, tertiary and/or quaternary amine groups. Examples of resins useful in chromatofocusing include the Mono™-P columns (poly(styrene divinylbenzene) cross-linked, grafted with secondary, tertiary and/or quaternary amine groups), PBE 94 and PBE 118 (cross-linked 6% agarose resins grafted with secondary, tertiary and/or quaternary amine groups bound to monosaccharides through ether bonds) marketed by GE Life Sciences or GE Healthcare. The Mono™-P and PBE 94 columns are suitable for separation between pH 9 and pH 4, while column PBE 118 is suitable for separation with a pH gradient beginning above pH 9. The Mono™-P and PBE 94 columns, and notably the column Mono™-P, are preferred. The initial buffers used may notably be based on a solution of diethanolamine, of Tris, of triethanolamine, of bis-Tris, of trielthylamine, of ethanolamine, of imidazole, of histidine, or piperazine at different pHs (addition of an HCl type acid, acetic acid, or iminodiacetic acid).

The elution amphoteric buffers used notably include the buffers Polybuffer 74 (pH range: 7-4, for the Mono™-P and PBE 94 columns), Polybuffer 96 (pH range: 9-6, for Mono™-P and PBE 94 columns), and Pharmalyte pH8-10.5 (pH range: 11-8, for the PBE 118 column).

Specific instructions of use and of selection of these buffers are available from the manufacturer of these columns.

Still another chromatography technique allowing separation of the charge isoforms of an antibody composition is hydrophobic interactions chromatography .

Thus, advantageously, in step b) of the method permitting to obtain a monoclonal antibody composition for use as a medicament according to the invention, the fractionation of step a) is achieved by one of the following chromatography techniques:
ion (anion or cation) exchange chromatography with elution by an ionic force gradient (ion exchange chromatography with an ionic force gradient),
ion (anion or cation) exchange chromatography with elution by a pH gradient (increasing in the case of cation exchange, decreasing in the case of anion exchange) (ion exchange chromatography with a pH gradient),
ion (anion or cation) exchange chromatography with elution by an ionic force and pH gradient (hybrid ion exchange chromatography),
ion (anion or cation) exchange chromatography with elution by a displacement molecule (displacement ion exchange chromatography),
chromatofocusing, and
hydrophobic interactions chromatography .

Advantageously, in step b) of the method permitting to obtain a monoclonal antibody composition for use as a medicament according to the invention, the fractionation of step a) is achieved by one of the following chromatography techniques:
ion exchange chromatography (regardless of the elution mode), in particular ion exchange chromatography with a pH gradient, and
chromatofocusing.

In particular, the inventors were able to separate the charge isoforms or variants of a monoclonal antibody composition with two different techniques, which may be used within the scope of the invention:
chromatofocusing: a column Mono™ P (GE Life Sciences) with elution by a decreasing pH gradient (from 9.5 to 8.0 by using two buffers: buffer A (diethanolamine 25 mM), buffer B (polybuffer 96 +pharmalyte 8-10.5));
cation exchange chromatography (column SCX, MabPac, Dionex) with elution by an increasing pH gradient (Buffer A: 20 mM NaH₂PO₄, 60 mM NaCl (pH 6); Buffer B: 20 mM Na₂HPO₄, 60 mM NaCl (pH10), gradient: 10% to 60% of Buffer B within 60 minutes).

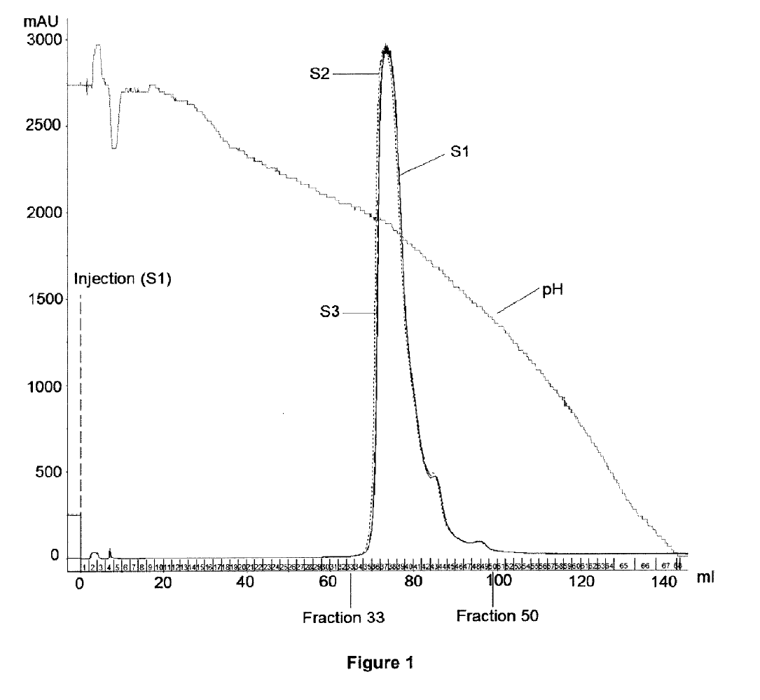

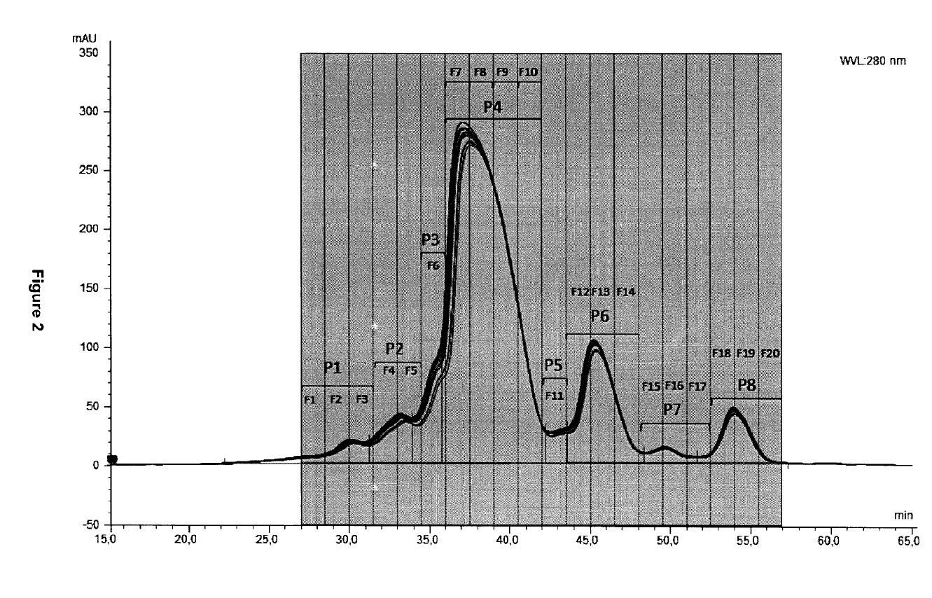

The chromatogram of an antibody composition obtained by a chromatography technique allowing separation of the charge isoforms always comprises a major peak comprising the major charge isoform as well as other isoforms close to the major isoform (i.e. with not many modifications relatively to the major isoform and therefore an pI and a net charge at a given pH very close to that of the major isoform), surrounded with minority peaks comprising so-called “acidic” isoforms on the one hand, the pI of which is inferior compared to the major isoform, and so- called “basic” isoforms on the other hand, the pI of which is superior compared to the major isoform (see FIGS. 1-2).

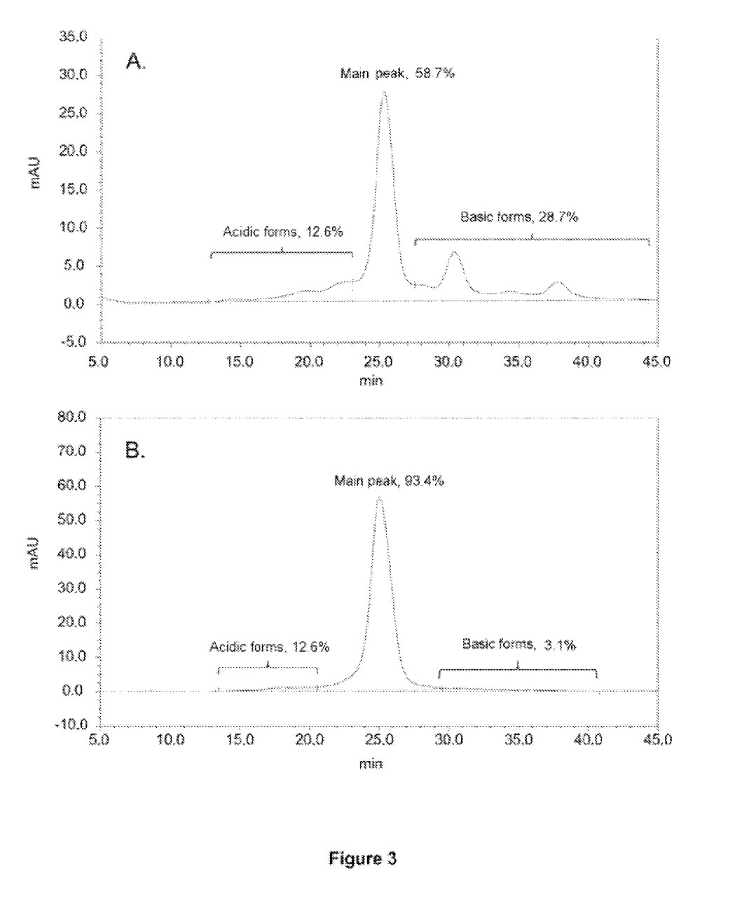

Depending on the chromatography technique used, the different isoforms appear on the chromatogram and are eluted in the following order:
Use of cation exchange chromatography (negatively charged resin), regardless of the elution mode (elution by an ionic force gradient, a pH gradient, a pH and ionic force gradient or by a displacement molecule): the acidic isoforms (which are less positively charged than the major isoform) are eluted the first, followed by the major isoform, and then the basic isoforms (which are more positively charged than the major isoform) (see FIG. 1 of Khawli et al.—2010, FIG. 3 of Rea et al.—2012; FIG. 1 of Farnan et al.—2009 and FIG. 1 of Rea et al.—2011; FIG. 1 of Zhang et al.—2011 and FIG. 8.10.2 of McAtee et al.—2012; and FIG. 2 of the present description);
Use of an anion exchange chromatography (positively charged resin), regardless of the elution mode (elution by an ionic force gradient, a pH gradient, a pH and ionic force gradient or by a displacement molecule): the basic isoforms (which are less negatively charged than the major isoform) are eluted the first, followed by the major isoform, and then by the acidic isoforms (which are more negatively charged than the major isoform);
Chromatofocusing: the basic isoforms are eluted the first, followed by the major isoform, and then by the acidic isoforms (see FIG. 1 of the present description).

The charge isoforms or variants of an antibody present within an antibody composition produced by a cell clone, a non-human transgenic animal or a transgenic plant, may also be separated with technologies other than chromatography. However, if these technologies are very useful with a purpose of analyzing or characterizing charge isoforms or variants, they do not allow separation of these isoforms with an acceptable yield and are therefore not very used with a preparative purpose.

Among such other technologies, mention may notably be made of isoelectric focusing (said to be “IEF” for “Isoelectric focusing”, and also called electrofocusing).

The basic principle of isoelectric focusing (IEF) is to generate in a gel (optionally included in a capillary) a pH gradient in which the proteins subjected to an electric field may move. The proteins will migrate in this electric field. Upon arriving at the pH corresponding to their pI, they will become immobilized since their net charge will be zero. In this way, it is possible to separate the proteins of a preparation according to their pI. It is possible to generate such a pH gradient with polyelectrolytes bearing a certain number of positively or negatively ionizable groups (amines, carboxyls or sulfates) and having a certain buffering capacity. These molecules are called ampholytes. If these ampholytes are subjected to an electric field limited by a solution of a strong acid at the anode and by a solution of a strong base at the cathode, they will migrate and be distributed by order of their pI. Their buffering capacity will contribute to maintaining around them a small pH area equal to their pI. A series of ampholytes each having an pI covering a certain pH range will therefore generate a continuous pH gradient. If a small amount of proteins in this system is caused to migrate, after or during its formation, they will also migrate and will be immobilized at their pI.

As an inert matrix for the gel, it is possible to use agarose, acrylamide or more rarely dextran, in which the pH gradient will be formed. A polyacrylamide gel is most often used. Since only the pI should influence the migration, concentrations of acrylamide has to be used, for which the porosity will not slow down the large proteins relatively to the small ones but which is sufficiently solid so as to be easily handled. A 5-6% gel is generally adequate.

The buffer of the anode is a strong acid, generally phosphoric acid. At the cathode, a strong base is placed, often triethanolamine.

The ampholytes are included in the mixture for preparing the gel before its polymerization. These molecules, which are polyelectrolytes, move in the electric field and are positioned following each other in the order of their own pI. Many companies make a large number of mixtures of ampholytes covering very narrow or very wide pH ranges: Ampholine® (notably Ampholine® pH 6/8 and Ampholine® pH 7/9 marketed by Sigma Aldrich), Pharmalyte® (notably Pharmalyte® pH 8/10.5 notably marketed by Sigma Aldrich and GE Healthcare, Life Sciences), BioLite® (notably BioLite® pH 6/8, BioLite® pH 7/9 and BioLite® pH 8/10 marketed by Bio-Rad), Zoom® (notably Zoom® pH 6/9 marketed by Life technologies/Invitrogen), Servalyt™ (notably Servalyt™ pH 6/8, Servalyt™ pH 6/9, Servalyt™ pH 7/9 marketed by Serva), SinuLyte™ (notably SinuLyte™ pH 6/8, SinuLyte™ pH 6/9, SinuLyte™ pH 7/9, SinuLyte™ pH 8/10 marketed by Sinus), etc. When a voltage is applied between both electrodes, each ampholyte will move as far as its isoelectric point and will become immobilized there. Gradients with various pH amplitudes may be generated by combining various ampholytes. In particular, for the analysis of charge isoforms in an antibody composition, gradients may be produced with very small intervals (e.g. 0.1 pH unit) between each ampholyte, on a small pH range centered on the average pI of the antibody and corresponding to the pI range of the different isoforms (for example between pH 6 and pH 8 or between pH 7 and pH 9), allowing a very fine separation of the different charge isoforms.

The antibody composition to be analyzed may be added after polymerization of the gel or directly in the mixture before polymerization. As the antibodies are larger than the ampholytes, they will migrate much more slowly and the ampholytes may therefore stabilize at their pI quite before substantial movement of the antibodies.

The migration time is not critical. Indeed, the antibodies do not risk leaving the gel when they will be immobilized at the point where they will have attained their pI. Only the migration should last for a sufficiently long time so that the ampholytes have the time of properly migrating and the antibodies have the time for attaining their pI. At 2 mA, the required time is estimated to be about 1 hour.

After migration, the gel may be colored for analyzing the different charge isoforms present in the antibody composition. The coloration may be achieved by any usual technique used in standard electrophoresis. However, the ampholytes should be removed from the gel since they may become colored. Therefore generally coloration is preceded by soaking in a 5 or 10% trichloroacetic acid bath or having them diffuse out of the gel while fixing the antibodies on site.

The use of markers having a given pI gives the possibility of quite specifically determining the pI of the different charge isoforms.

Following coloration, the proportion in the analyzed composition of each charge isoform separated in IEF relatively to the total isoforms may be quantified by means of image analysis software packages, such as the software package Quantity One® for example, marketed by Bio-Rad.

Although very accurate and sensitive for separating the charge isoforms present in an antibody composition, the isoelectric focusing technology does not give the possibility of easily harvesting the separated isoforms and is therefore generally used rather for purposes of analysis and of quantification than for the purpose of preparative separation of the different isoforms.

In step c) of the method, the composition of interest according to the invention, intended to be used as a medicament, is obtained by combining one or several chromatographic fractions obtained in step b), corresponding to the major peak of the chromatogram, the thereby obtained monoclonal antibody composition being enriched in said major peak, the latter representing at least 85%, advantageously at least 86%, at least 87%, at least 88%, at least 89%, more advantageously at least 90%, at least 91%, at least 92%, at least 93%, at least 94%, or even at least 95%, at least 96%, at least 97%, at least 98%, at least 98.5%, at least 99%, or at least 99.5% of the chromatogram of the composition obtained in step c).

Advantageously, in a composition for use as a medicament according to the invention, at least 95%, advantageously at least 96%, at least 97%, at least 98%, or even at least 98.5%, at least 99%, or at least 99.5% of the heavy chains of the antibodies present in the composition do not comprise any C-terminal lysine residue.

The invention also relates to a monoclonal antibody composition, wherein at least 95%, advantageously at least 96%, at least 97%, at least 98%, or even at least 98.5%, at least 99%, or at least 99.5% of the heavy chains of the antibodies present in the composition do not comprise any C-terminal lysine residue, for its use as a medicament. Indeed, the basic isoforms of the antibodies present in the composition have at least one heavy chain with a C-terminal lysine residue. Such a composition therefore exclusively comprises the major isoform and the acidic isoforms. As the basic isoforms are not very active for the effector functions via FcγRIII and via the complement (see Examples) and represent about 8 to 20% before purification (as measured by chromatography), such a composition is capable of inducing stronger ADCC via FcγRIII and a stronger response CDC than the total composition, before exclusion of basic isoforms. Such a composition may be obtained by chromatographic separation as described above, the collected fractions however corresponding in this case to that of acidic and major isoforms.

The antibody composition that may be obtained by the method described above and that is intended to be used as a medicament, may be used in any pathology that may be treated with monoclonal antibodies, in particular when the destruction of target cells by ADCC or by CDC is useful for the treatment.

Today it is known that ADCC is an essential mechanism for the clinical efficiency of a passive immunotherapy treatment by means of antibodies intended to treat cancers (Wallace et al.—1994; Velders et al.—1998; Cartron et al.—2002; Ianello et al.—2005; Weiner et al.—2010), to prevent allo-immunization in Rhesus-negativepregnant women (Béliard et al.—2008). Further, the ADCC response is also known for playing a significant role in the anti-infectious response against viruses (Ahmad et al.—1996, Miao et al.—2009), bacteria (Albrecht et al.—2007; Casadevall et al.—2002) and parasites (Zeitlin et al.—2000). Further, in the context of of autoimmune diseases, new therapies aim at removing the immune cells responsible for the attacks, such as the B or T lymphocytes for example, ADCC then playing a highly significant role (Edwards et al.—2006; Chan et al.—2010). The CDC response is also known for being significant in various pathologies and notably in the treatment of cancers.

Thus, in the compositions for use as a medicament according to the invention, the antibody is advantageously directed against a non-ubiquitous antigen present on the healthy donor cells, an antigen of a cancer cell, an antigen of a cell infected by a pathogenic agent, or an antigen of an immune cell.

In particular, the following embodiments are preferred:
the antibody is an anti-Rhesus (D) antibody (notably Roledumab,

Atorolimumab or Morolimumab, in particular Roledumab) and the composition is intended for preventing allo-immunization in Rhesus-negative individuals,
the antibody is directed against an antigen of a cancer cell and the composition is intended for treating a cancer,
the antibody is directed against an antigen of a cell infected by a pathogenic agent and the composition is intended for treatment of an infection by said pathogenic organism,
the antibody is directed against an antigen of an immune cell and the composition is intended for treating an autoimmune disease.

IN the context of the treatment of cancers, the antibodies may notably be directed against the following antigens: CD20, Her2/neu, CD52, EGFR, EPCAM, CCR4, CTLA—4 (CD152), CD19, CD22, CD3, CD30, CD33, CD4, CD40, CD51 (Integrin alpha-V), CD80, CEA, FR-alpha, GD2, GD3, HLA-DR, IGF1R (CD221), phosphatidylserine, SLAMF7 (CD319), TRAIL-R1, TRAIL-R2.

More specifically, specific (antigen/cancer) pairs known for their therapeutic interest (antibodies of this antigen specificity approved in at least one country for treatment of the mentioned cancer, or clinical trials being conducted) are indicated in Table 1 below.

TABLE 1
Specific (antigen/cancer) pairs of interest

| | Example of an antibody | Cancer(s) which may be treated with |
|---|---|---|
| Antigen | directed against this antigen | an antibody of this antigen specificity |
| CD20 | rituximab, ofatumumab, | Haematologic cancers, notably: |
| | Ocrelizumab, Tositumomab, | non-Hodgkin lymphoma, B-cell |
| | Veltuzumab, | lymphoma, chronic lymphocyte |
| | Ublituximab | leukaemia, follicular lymphoma, |
| Her2/neu | Trastuzumab, Pertuzumab | Solid cancers, notably: Breast |
| | | cancer, lung cancer not with small |
| | | cells, pancreas cancer, prostate |
| | | cancer, ovary cancer |
| CD52 | alemtuzumab | Haematological cancers, notably: |
| | | Chronic lymphocyte leukaemia, |
| | | chronic myeloid leukaemia, |
| | | cutaneous or peripheral T-cell |
| | | lymphoma |
| EGFR | Cetuximab, panitumumab, | Solid tumours, notably: Colorectal |
| | Futuximab, Imgatuzumab, | cancer, head and neck cancer, lung |
| | Matuzumab, Necitumumab, | cancer, oesophagus cancer, |
| | Nimotuzumab, | stomach cancer, glioma, anaplasic |
| | Zalutumumab | astrocytoma, glioblastoma |
| EPCAM | Edrecolomab, | Solid cancers, notably: Colorectal |
| | Adecatumumab, Solitomab, | cancer, prostate cancer, breast |
| | | cancer |
| CCR4 | Mogamulizumab | Haematological cancers, notably: |
| | | Adult T-cell leukaemia/lymphoma |
| CTLA-4 | Ipilimumab, Tremelimumab, | Solid tumours, notably: melanoma, |
| (also known | | prostate cancer, bladder cancer |
| under the | | |
| name of | | |
| CD152) | | |
| CD19 | Blinatumomab (targets both | Haematologic cancers, notably: |
| | CD19 and CD3) | non-Hodgkinien lymphoma, acute |
| | | lymphoblastic leukaemia, lung |
| | | cancer, gastrointestinal cancer |
| CD22 | Epratuzumab | Haematologic cancers, notably: B- |
| | | cell cancers |
| CD3 | Otelixizumab, Teplizumab, | Haematologic cancers, notably: |
| | Visilizumab | multiple myeloma |
| CD30 | Iratumumab | Haematologic cancers, notably: |
| | | non-Hodgkinien lymphoma |
| CD33 | Lintuzumab | Haematologic cancers, notably: |
| | | acute myeloid leukaemia, |
| | | myelodysplasic syndromes |
| CD4 | Cedelizumab, Clenoliximab, | Melanoma, cutaneous or peripheral |
| | Priliximab, Zanolimumab | T-cell lymphoma |
| CD40 | Dacetuzumab, | Haematologic cancers, notably: |
| | Lucatumumab, Teneliximab | non-Hodgkinien lymphoma, Hodgkin |
| | | lymphoma, multiple myeloma |
| CD51 | Intetumumab | Solid tumours |
| (Integrin | | |
| alpha-V) | | |
| CD80 | Galiximab | Haematologic cancers, notably: B- |
| | | cell lymphoma |
| CEA | Labetuzumab | Solid tumours, notably: colorectal |
| | | cancer |
| FR-alpha | Farletuzumab | Ovary cancer |
| Ganglioside | 3F8, TRBS07 | Neuroblastoma, melanoma |
| GD2 | | |
| Ganglioside | Ecromeximab, Mitumomab | Melanoma, lung cancer with small |
| GD3 | | cells |
| HLA-DR | Apolizumab | Haematologic cancers |
| IGF1R | Cixutumumab, | Solid tumours, notably: lung cancer |
| (CD221) | Figitumumab, | not with small cells, adenocortical |
| | Robatumumab, Ganitumab | carcinoma, pancreas cancer |
| phosphatidyl | Bavituximab | Solid tumours, notably: breast |
| serine | | cancer, lung cancer not with small |
| | | cells |
| SLAMF7 | Elotuzumab | Multiple myeloma |
| (CD319) | | |
| TRAIL-R1 | Mapatumumab | Solid tumours, notably: lung cancer |
| | | not with small cells, colorectal |
| | | cancer; non-Hodgkinien lymphoma |
| TRAIL-R2 | Conatumumab, | Solid tumours, notably: breast |
| | Lexatumumab, | cancer, pancreas cancer, colorectal |
| | Tigatuzumab | cancer, lung cancer not with small |
| | | cells, ovary cancer |

In the context of the treatment of infections by pathogenic organisms, the antibodies may notably be directed against the following antigens: antigens of Clostridium difficile, antigens of Staphylococcus aureus (notably ClfA and lipotheicoic acid), antigens of the cytomegalovirus (notably glycoprotein B), antigens of Escherichia coli (notably Shiga-like toxin, under unit IIB), antigens of the syncytial respiratory virus (Protein F notably), antigens of the hepatitis B virus, antigens of the A Influenza virus (Hemagglutinin notably), antigens of Pseudomonas aeruginosa of serotype IATS O11, antigens of rabies viruses (Glycoprotein notably), phosphatidylserine.

More specifically, specific (antigen/infectious disease) pairs known for their therapeutic interest (antibody of this antigen specificity approved in at least one country for treating the mentioned infectious disease, or clinical trials in progress) are indicated in Table 2 below.

TABLE 2
Specific (antigen/infectious disease) pairs of interest

| | Example of an antibody | Infectious disease(s) which may |
|---|---|---|
| | directed against this | be treated with an antibody of this |
| Antigen | antigen | antigen specificity |
| Antigen of | Actoxumab, | Clostridium difficile infection |
| Clostridium difficile | Bezlotoxumab | |
| ClfA antigen of | Tefibazumab | Staphylococcus aureus infection |
| Staphylococcus | | |
| aureus | | |
| Antigen of the | Sevirumab | Infection by the cytomegalovirus |
| cytomegalovirus | | |
| Glycoprotein B of | Regavirumab | Infection by the cytomegalovirus |
| the | | |
| cytomegalovirus | | |
| Shiga-like toxin, | Urtoxazumab | Infection by Escherichia coli, |
| sub unit IIB of | | serotype O121 |
| Escherichia coli | | |
| Protein F of the | Palivizumab, | Infection by the syncytial |
| syncytial | Motavizumab | respiratory virus |
| respiratory virus | | |
| Surface antigen of | Exbivirumab, Libivirumab | Infection by the hepatitis B virus |
| the hepatitis B | | |
| virus | | |
| Antigen of the | Tuvirumab | Infection by the hepatitis B virus |
| hepatitis B virus | | |
| Haemagglutinin of | CR6261 | Influenza, notably Spanish |
| the Influenza A | | influenza and H5N1 |
| virus | | |
| Lipotheicoic acid | Pagibaximab | Staphylococcus aureus infection, |
| of Staphylococcus | | septic shock by Staphylococcus |
| aureus | | aureus |
| phosphatidylserine | Bavituximab | Infection by viruses of hepatitis C, |
| | | influenza A and B, HIV 1 and 2, |
| | | German measles, respiratory |
| | | syncytial virus, pichinde virus |
| Antigen of | Panobacumab | Infection by Pseudomonas |
| Pseudomonas | | aeruginosa |
| aeruginosa | | |
| serotype IATS | | |
| O11 | | |
| Glycoprotein of | Foravirumab, | Infection by the rabies virus |
| the virus of rabies | Rafivirumab | |

In the context of the treatment of autoimmune diseases, the antibodies may notably be directed against the following antigens: CD20, CD52, CD25, CD2, CD22, CD3, and CD4.

More specifically, specific (antigen/autoimmune disease) pairs known for their therapeutic interest (antibody of this antigen specificity approved in at least one country for treating the mentioned autoimmune disease, or clinical trials in progress) are indicated in Table 3 below.

TABLE 3
Specific (antigen/autoimmune disease) pairs of interest

| | Example of | Autoimmune disease(s) which may |
|---|---|---|
| | antibodies directed | be treated with an antibody of this |
| Antigen | against this antigen | antigen specificity |
| CD20 | rituximab, ofatumumab, | Rheumatoid arthritis, |
| | Ocrelizumab, | thrombocytopenic purpura, lupus |
| | Tositumomab, | erythematosus, multiple sclerosis |
| | Veltuzumab, | |
| | Ublituximab | |
| CD52 | alemtuzumab | Multiple sclerosis |
| CD25 | Daclizumab, Basiliximab, | Uveitis, multiple sclerosis, |
| | Inolimomab | psoriasis, diabetes of type 1, |
| | | ulcerative colitis |
| CD2 | Siplizumab | psoriasis |
| CD22 | Epratuzumab | lupus erythematosus |
| CD3 | Otelixizumab, Teplizumab, | Diabetes of type 1, ulcerative |
| | Visilizumab | colitis, Crohn's disease |
| CD4 | Cedelizumab, | Rheumatoid arthritis, Crohn's |
| | Clenoliximab, | disease, multiple sclerosis, |
| | Priliximab, | psoriasis |
| | Zanolimumab | |

The antibody compositions intended for use as a medicament according to the invention are notably intended for therapies implying an ADCC response, which includes many scenarios as explained in detail above. It is therefore advantageous that these antibodies have also been optimised by other means for inducing an ADCC response in vivo via FcγRIII receptor, as strong as possible. Thus, in an advantageous embodiment, in a composition for a use as a medicament according to the invention, the antibody comprises a modification of the Fc fragment enhancing its binding to FcγRIII receptor and its effector properties via FcγRIII receptor. Two main means have for the moment been described for optimising ADCC activity via FcγRIII receptor:
Insertion of at least one mutation at certain amino acid residues of the Fc fragment, as notably described in WO00/42072, Shields et al.—2001, Lazar et al.—2006, WO2004/029207, WO/2004063351, WO2004/074455.
Optimisation of the nature of the N-glycans attached to the Asn297 residue of each heavy chain in the Fc fragment.

Thus, in an advantageous embodiment, a composition for use as a medicament according to the invention comprises a monoclonal antibody, the sequence of which has been modified at least at one amino acid residue of the Fc fragment for enhancing the binding to the FcγRIII receptor, as described in WO00/42072, Shields et al.—2001, Lazar et al.—2006, WO2004/029207, WO/2004063351, WO2004/074455.

In particular, mutations at the following positions of Fc were described as allowing an increase in the affinity for the FcγRIII receptor and the capability of inducing ADCC via this receptor: 219, 222, 224, 239, 247, 256, 267, 270, 283, 280, 286, 290, 294, 295, 296, 298, 300, 320, 326, 330, 332, 333, 334, 335, 339, 360, 377, 396. More particularly, the following substitutions were described as permitting to increase the affinity for the FcγRIII receptor and the capability of inducing ADCC via this receptor: S219Y; K222N; H224L; L234E, L234Y, L234V; L235D, L235S, L235Y, L2351; S239D, S239T; V2401, V240M; P247L; T256A, T256N; V2641, V264T; V2661; S267A; D270E; D280A, D280K, D280H, D280N, D280T, D280Q, D280Y; V282M; E283Q; N286S; K290A, K290Q, K290S, K290E, K290G, K290D, K290P, K290N, K290T, K290S, K290V, K290T, K290Y; E294N; Q295K; Y296W; S298A, S298N,

S298V, S298D, S298E; Y3001, Y300L; K320M, K320Q, K320E; N325T; K326S, K326N, K326Q, K326D, K326E; A330K, A330L, A330Y, A3301; 1332E, 1332D; E333A, E333Q, E333D; K334A, K334N, K334Q, K334S, K334E, K334D, K334M, K334Y, K334H, K334V, K334L, K3341; T335E, T335K; A339T; K360A; F372Y; 1377F; V379M; P396H, P396L; D401V.

Combinations of interesting mutations include: E333A/K334A, T256A/S298A, S298A/E333A, S298A/K334A, S298A/E333A/K334A, S267A/D280A (WO00/42072), S239D/I332E, S239D/1332E/A330L (Lazar et al.—2006), V2641/1332E, S298A/I332E, S239E/I332E, S239Q/I332E, S239D/I332D, S239D/I332E, S239D/1332N, S239D/I332Q, S239E/I332D, S239E/1332N, S239N/I332E, S239Q/I332D,

A330Y/1332E, V2641/A330Y/1332E, A330L/1332E, V2641/A330L/1332E, S239E/V2641/1332E, S239E/V2641/A330Y/1332E, S239D/A330Y/1332E, S239N/A330Y/1332E, S239D/A330L/1332E, S239N/A330L/1332E, V2641/S298A/1332E, S239D/S298A/1332E, S239N/S298A/1332E, S239D/V2641/1332E (WO2004/029207).

Alternatively or additionally, a monoclonal antibody composition for use as a medicament according to the invention comprises a low fucose content. By “fucose content”, is meant the percentage of fucosylated forms within the N-glycans attached to the Asn297 residue of the Fc fragment of each heavy chain of each antibody. By “low fucose content” is meant a fucose content of less than or equal to 65%. Indeed, it is today known that the fucose content of an antibody composition plays a crucial role in the capability of this composition of inducing a strong ADCC response via the FcγRIII receptor. Advantageously, the fucose content is less than or equal to 65%, preferably less than or equal to 60%, 55% or 50%, or even less than or equal to 45%, 40%, 35%, 30%, 25% or 20%. However, it is not necessary that the fucose content be zero, and it may for example be greater than or equal to 5%, 10%, 15% or 20%. The fucose content may for example be comprised between 5 and 65%, between 5 and 60%, between 5 and 55%, between 5 and 50%, between 5 and 45%, between 5 and 40%, between 5 and 35%, between 5 and 30%, between 5 and 25%, between 5 and 20%, between 10 and 65%, between 10 and 60%, between 10 and 55%, between 10 and 50%, between 10 and 45%, between 10 and 40%, between 10 and 35%, between 10 and 30%, between 10 and 25%, between 10 and 20%, between 15 and 65%, between 15 and 60%, between 15 and 55%, between 15 and 50%, between 15 and 45%, between 15 and 40%, between 15 and 35%, between 15 and 30%, between 15 and 25%, between 15 and 20%, between 20 and 65%, between 20 and 60%, between 20 and 55%, between 20 and 50%, between 20 and 45%, between 20 and 40%, between 20 and 35%, between 20 and 30%, between 20 and 25%.

The antibody composition may moreover have different types of glycosylation (N-glycans of the oligomannose or biantennary complex type, with a variable proportion of bisecting N-acetylglucosamine (GIcNAc) residues or galactose residues in the case of N-glycans of the biantennary complex type), provided that they have a low fucose content. Thus, N-glycans of the oligomannose type may be obtained by cultivation in the presence of different glycosylation inhibitors, such as inhibitors of α1,2-mannosidase I (like Deoxymannojirimycin or “DMM”) or α-glucosidase (like castanospermin or “Cs”); or else by producing the antibody in the CHO Lec 1 line. Production in the milk of transgenic goats also leads to obtaining antibodies for which the major N-glycan is of the oligomannose type, with as minority forms fucosylated biantennary complex forms with one or two galactoses, without any bisecting GlcNAc and without sialylation (G1F or G2F) (see WO2007048077A2). N-glycans of the biantennary complex type may be obtained in most mammal cells, but also in bacteria, yeasts, or plants, the glycosylation machinery of which has been modified. In order to limit the fucose content, cell lines naturally having low activity of the enzyme FUT8 (1,6-fucosyltransferase) responsible for the addition of fucose on the GIcNAc bound to the Fc fragment; such as the cell line YB2/0, the duck embryo cell line EB66®, or the rat hepatoma cell lines H4-II-E (DSM ACC3129), H4-II-Es (DSM ACC3130); may be used. Cell lines mutated for other genes and the sub-expression or over-expression of which leads to a low fucose content may also be used, like the CHO Lec13 cell line, a mutant of the CHO cell line having a reduced synthesis of GDP-fucose. It is also possible to select a cell line of interest and to decrease or abolish (notably by using interfering RNAs or by mutation or deletion of the gene expressing the protein of interest) the expression of a protein involved in the fucosylation route of N-glycans (notably FUT8, see Yamane-Ohnuki et al.—2004; but also GMD, a gene involved in the transport of GDP-fucose, see Kanda et al.—2007). Another alternative consists in selecting a cell line of interest and in over-expressing a protein somehow interfering with the fucosylation of N-glycans, like the protein GnTIII (β(1,4)-N-acetylglucosaminetransferase III). In particular, antibodies having slightly fucosylated N-glycans were notably obtained by:
Production in YB2/0 (see EP1176195A1, WO01/77181, Shinkawa et al.—2003), CHO Lec13 (see Shields et al.—2002), EB66® (Olivier et al.—2010), or rat hepatoma lines H4-II-E (DSM ACC3129), H4-II-Es (DSM ACC3130) (see WO2012/041768).
Production in a wild type CHO cell line in the presence of small interfering RNAs directed against FUT8 (Mori et al.—2004, Suzuki et al.—2007, Cardarelli et al.—2009, Cardarelli et al.—2010, Herbst et al.—2010), or GMD (gene coding for the transporter of GDP-fucose in the Golgi apparatus, see Imai-Nishiya et al.—2007)
Production in a CHO cell line, of which the two alleles of the gene FUT8 encoding 1,6-fucosyltransferase have been deleted (Yamane-Ohnuki et al.—2004), or of which both alleles of the GMD gene encoding the transporter of GDP-fucose in the Golgi apparatus have been deleted (Kanda et al.—2007),
Production in a CHO cell line in which the gene encoding the enzyme GnTIII (β(1,4)-N-acetylglucosaminetransferase III) was over-expressed by transgenesis (Umana et al.—1999). In addition to low fucosylation, the N-glycans obtained are characterised by a strong content of bisecting GIcNAc.
Production in transgenic plants (N. benthamiana), with a strong reduction of the contents of β1,2-xylose and α1,3-fucose residues by means of the use of small interfering RNAs (Forthal et al.—2010).

The N-glycans of the oligomannose type have reduced half-life in vivo as compared with N-glycans of the biantennary complex type. Consequently, advantageously, the antibodies present in the composition have on their N-glycosylation sites of the Fc fragment glycan structures of the biantennary complex type, with a low fucose content, as defined above.

In particular, the monoclonal antibody composition may have a content of G0+G1+G0F+G1F forms greater than 60% and a low fucose content as defined above. It may also have a content of G0+G1+G0F+G1F greater than 65% and a low fucose content, as defined above. It may also have a content of G0+G1+G0F+G1F of more than 70% and a low fucose content, as defined above. It may also have a content of G0+G1+G0F+G1F of more than 75% and a low fucose content, as defined above. It may also have a content of G0+G1+G0F+G1F forms of more than 80% and a low fucose content, as defined above. It may also have a content of G0+G1+G0F+G1F forms of more than 60%, 65%, 70%, 75% or 80% and a content of G0F+G1F forms of less than 50%. The forms GO, G1, GOF and G1F are as defined below:

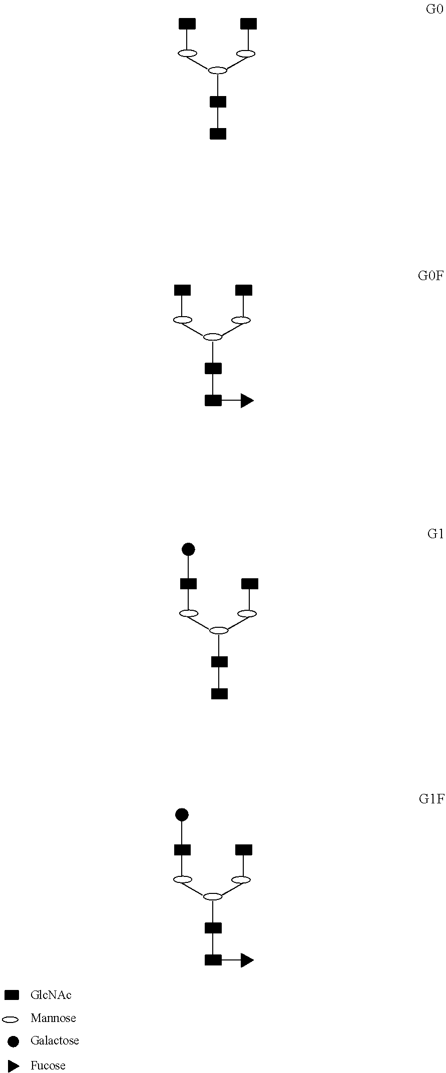

Such antibody compositions may notably be obtained by production in YB2/0, in CHO Lec13, in wild-type CHO cell lines cultivated in the presence of small interfering RNAs directed against FUT8 or GMD, in CHO cell lines for which both alleles of the gene FUT8 encoding 1,6-fucosyltransferase or both alleles of the gene GMD encoding the transporter of GDP-fucose in the Golgi apparatus have been deleted.

The antibody compositions intended for use as a medicament according to the invention are also intended for therapies involving a CDC response. It may therefore be also advantageous, additionally or alternatively to modifications increasing the activity via FcγRIII that these antibodies have also been optimised by other means for inducing a CDC response in vivo via the protein C1q as strong as possible. Thus, in an advantageous embodiment, in a composition for use as a medicament according to the invention, the antibody comprises a modification of the Fc fragment enhancing its binding to the protein C1q and its effector properties via the complement.

Such mutations are notably described in the following documents: WO2004074455A2, Idusogie et al.—2001, Dall'Acqua et al.—2006(b), and Moore et al.—2010.

The present invention also relates to the use of a chromatography fractionation step in order to increase the ability of a monoclonal antibody composition directed against a given antibody to induce antibody-dependent cell cytotoxicity (ADCC) of target cells expressing said antigen by the effector cells of the immune system expressing the FcγRIII (CD16)receptor.

The thereby obtained composition has improved ability to induce ADCC of target cells expressing the antigen of interest by the effector cells of the immune system expressing the FcγRIII (CD16)receptor, and notably by natural killer cells (or NK cells). Preferably, the ratio R of the ADCC levels obtained with the composition enriched in isoforms of the major peak and with the composition before fractionation, defined by the following formula:

$$R = \frac{\begin{matrix}ADCC level obtained with the composition enriched \\ in isoforms of the major peak\end{matrix} }{\begin{matrix}ADCC level obtained with \\ the composition before fractionation\end{matrix}}$$

is of at least 1.15 (corresponding to an increase in the ADCC level of at least 15%); advantageously at least 1.16; at least 1.17; at least 1.18; at least 1.19; more advantageously at least 1.20; at least 1.25; at least 1.30; at least 1.35; at least 1.40; at least 1.45; or even at least 1.50 (corresponding to an increase in the ADCC level of at least 50%).

The present invention also relates to the use of a chromatography fractionation step for increasing the ability of a monoclonal antibody composition directed against a given antibody to induce complement-dependent cytotoxicity (CDC) of target cells expressing said antigen by the complement.

The thereby obtained composition has improved ability to induce lysis by the complement of target cells expressing the antigen of interest. Preferably, the ratio R of the CDC levels obtained with the composition enriched in isoforms of the major peak and with the composition before fractionation, defined by the following formula:

$$R = \frac{\begin{matrix}CDC level obtained with the composition enriched \\ in isoforms of the major peak\end{matrix} }{\begin{matrix}CDC level obtained with \\ the composition before fractionation\end{matrix}}$$

is of at least 1.15 (corresponding to an increase of the CDC level of at least 15%); advantageously at least 1.16; at least 1.17; at least 1.18; at least 1.19; more advantageously at least 1.20; at least 1.25; at least 1.30; at least 1.35; at least 1.40; at least 1.45; or even at least 1.50 (corresponding to an increase in the CDC level of at least 50%).

In both uses above, the chromatography fractionation step may be carried out in any way described above for obtaining the antibody compositions enriched in major isoform for use as a medicament according to the invention. In particular, the fractionation may be carried out by one of the following chromatography techniques:
ion exchange chromatography, regardless of the elution mode (ionic force gradient, pH gradient, pH and ionic force gradient, displacement molecule);
chromatofocusing;
hydrophobic interactions chromatography .

The monoclonal antibody composition for which such a chromatography fractionation step is carried out with the purpose of increasing the ADCC or CDC response abilities via the effector cells expressing CD16 may be any monoclonal antibody composition described above. In particular, the monoclonal antibody present in the composition may be human, humanized or chimeric.

It may also be directed against any type of antigen and notably those described above. In particular, when the target cells are cancer cells, the antibody may be directed against a cancer cell antigen, and notably one of the antigens described above in the context of treating cancers. When the target cells are cells infected by a pathogenic agent, the antibody may be directed against an antigen of the infected cells, and notably against one of the antigens described above in the context of the treatment of infectious diseases. When the target cells are immune cells involved in the development of an autoimmune disease, the antibody may be directed against an antigen of these immune cells, and notably against one of the antigens described above in the context of the treatment of autoimmune diseases.

The chromatography fractionation step (step a) is preferably followed by a step of combining the obtained chromatographic fractions corresponding to the major peak of the chromatogram (step b), the thereby obtained monoclonal antibody composition being enriched in said major peak, the latter representing at least 85% of the chromatogram of the composition obtained in step b) (after fractionation and combination of the chromatographic fractions of interest).

The following examples correspond to illustrations of the present invention.

### EXAMPLES

### Example 1

### Preparation of Purified Fractions of the Charge Isoforms of an Anti-CD20 Antibody Composition, Characterisation of the Isoforms and of Their Effector Properties

### Equipment and Methods

### Anti-CD20 Antibody Composition

All the separations and analyses were carried out on a batch of an anti-CD20 antibody composition produced by a clone YB2/0.

### Separation of the Charge Isoforms by Chromatofocusing

Three preparative separations of charge isoforms of a same antibody composition were carried out by chromatofocusing.

An anion exchange resin Mono P 5/200 GL was used. 20 mg of salted-out protein were injected at each separation. The elution was carried out by a decreasing pH gradient (pH 9.5 to 8.0), by using the two following buffers:
Buffer A: diethanolamine 25 mM,
Buffer B: polybuffer 96 +pharmalyte 8-10.5.

The eluates of the separations were collected in 2mL fractions. The fractions of interest are the fractions 33 to 50.

The fractions of the 3 separations were concentrated for analysis.

The separation 1 (S1) was subject to a particular concentration so that the fractions may be made sterile by filtration:
Concentration of the fractions on Amicon Ultra 10 kDa for obtaining a volume of 1 mL
Sterilizing filtration
Sampling of an aliquot for measuring the concentration and of an aliquot for measuring the activity

### Separation of the Charge Isoforms by Cation Exchange Chromatography (CEX)

Eleven separations of charge isoforms were achieved by cation exchange chromatography with elution by an increasing pH gradient (CEX).

A cation exchange resin SCX (MabPac SCX 10.4×250 mm, Dionex) was used at 30° C. The elution was achieved by means of an increasing pH gradient (pH 6 to 10), by using both following buffers:
Buffer A: 20 mM NaH₂PO₄, 60 mM NaCl (pH 6),
Buffer B: 20 mM NaH₂PO₄, 60 mM NaCl (pH10).

The gradient was obtained in the following way: 10% to 60% of buffer B within 60 minutes.

The eluates of the separations were collected in fractions. The fractions of interest are the fractions 1 to 20.

### Analysis of the Binding to the CD16 Receptor by BIACORE

A method was developed for measuring the capability of an antibody composition of binding to the receptor CD16a by using the SPR (“Surface plasmon resonance”) technology on a Biacore T100 system (GEHealthcare). A soluble receptor CD16a was immobilised on the detection chip by using amine coupling. A flow cell is used for the antibody, the other flow cell is left free in order to subtract the background noise. The antibodies are injected at three concentrations and the kinetic parameters are estimated by producing for each concentration a binding ratio both to the association phase and to the dissociation phase. The SPR signal, expressed in resonance units (RU), represents the association and the dissociation of the antibody at the receptor.

### Activation of Effector Cells via CD16 (CD16 Jurkat Test)

The capability of various fractions separated by chromatofocusing and by cation exchange chromatography (CEX), comprising different charge isoforms, of inducing a response of effector cells via the CD16 receptor (FcγRIII) was tested. The test used is the following:

The antibodies are incubated with WIL2-S cells (positive CD20 target cells) and CD16 Jurkat cells (effector cells) (genotype CD16FF). The amount of cytokines (IL2) secreted by the CD16 Jurkat cells was measured by ELISA.

More specifically, in a 96-well plate, are mixed:
Antibody: 50 μl of dilutions ranging from 0.156 to 10 ng/ml in IMDM 5% FCS
PMA 50 μl of a dilution at 40 ng/ml in IMDM 5% FCS (i.e. 2ng PMA /50 μl)
WIL2-S cells: 50 μl at 6×10⁵/ml in IMDM 5% FCS (i.e. 30×10³ cells/50μl)
CD16 Jurkat: 50 μl at 10×10⁶/ml in IMDM 5% FCS (i.e. 500×10³cells/50μl)

Two controls are used: a negative control without any target cells and a positive control with maximum activity:

Negative control without any cells: are added per well:
50 μl of CD16 Jurkat cells at 10×10⁶cells/ml (i.e. 500×10³cells/50μl)
50 μl of PMA at 40 ng/ml (i.e. 2 ng PMA/50μl)
50 μl of antibody at the highest concentration
50 μl of IMDM medium +5% FCS

Maximum activity positive control: are added per well:
50 μl of CD16 Jurkat cells at 10×10⁶cells/ml (i.e. 500×10³ cells/50μl)
50 μl of PMA at 40 ng/ml (i.e. 2ng PMA/50μl)
50 μl of Ionomycin at 5 μg/ml
50 μl of IMDM medium +5% SVF

Gently stir and incubate for one night at 37° C. +/−0.5° C.

Decant the cells for 1 minute at 125 g

Transfer 160 μl of supernatant into a 96-well plate with round bottoms

Again decant the cells for 1 minute at 125 g

Dose IL-2 in the supernatant. Read out at 450 nm.

The CD16 activity (secretion of IL-2) of each sample is expressed as a percentage of the CD16 activity of a reference sample.

### Complement-Dependent Cytotoxicity (CDC)

The target cells Wil2-S are cultivated in a de-complemented IMDM medium with 10% of FCS (medium 110). They are transplanted twice a week into 100ml of media with 0.2 10⁶ cells/ml in a flask F175. The test is conducted on transplanted cells since 24 to 72 hours, and taken up again at 1.10⁶ cells/ml in a de-complemented medium IMDM+5% FCS (medium 15).

Human serum (human serum AB obtained by coagulation of full blood) is defrosted the day when it is used. Defrosting is carried out at +4° C. After defrosting, the serum is diluted to ½ in medium 15.

The CellTiter-Blue® (Promega) is stored at —20° C., it is left to defrost at room temperature before use.

The concentration of the antibodies to be studied is adjusted to 1 μg/ml in an 15 medium.

In a 96-well plate with U bottoms, add:
50 μl of target cells (Wil2S at 1.10⁶ cells/ml)
50 μl of antibody to be tested
50 μl of ½ diluted human serum.

The cells are directly deposited in the plate after adjustment to 1.10⁶C/ml and put at 37° C.

The cells are incubated for 5 minutes and the sample is stirred at 37° C. before depositing the serum.

Two controls are made: without any cells (C-) and with antibodies (AC-). The missing element is replaced with 15 medium.

They are incubated at 37° C. for 2 hours with stirring. Then 30 μl of CellTiter-Blue® are then added into each well, homogenisation is performed by reverse pipetting upon addition and incubation is performed at 37° C. for 3 hours and 30 minutes with stirring.

At the end of the incubation, the read out may be deferred to the next day by stopping and stabilising the reaction by adding 25 μl of 3% SDS. The plate is then kept at room temperature.

At the end of the incubation or the next day, the plates are centrifuged for 2 min at 125 g. A 100 μl of each well is sampled and then distributed in a black optical plate with transparent bottoms while retaining the plate plane.

The read out of the plate is carried out with the fluorescence reader with the following parameters:
Excitation: 530/25 nm
Emission: 590/20 nm
Read out through the bottom of the plate (FOND)
Integration time: 20 μs
Number of flashes: 25
Gain: calculated from the well taken in the control well not containing any antibodies (cells+serum+15 medium)
Stirring of intensity 2 for 15 seconds in an orbital mode

### Characterisation of the Isoforms by Mass Spectrometry

The various charge isoforms present in the various fractions separated by chromatofocusing or by cation exchange chromatography (CEX) were analysed by mass spectrometry as described in Chevreux—2011.

This method comprises the use of a bacterial protease cysteine (IdeS, an enzyme degrading immunoglobulins of Streptococcus pyogenes), which specifically cleaves the IgGs under their boundary domain, the heavy chain being cleaved into two fragments of 25 kDa respectively consisting of the VH-CH1 and CH2-CH3 domains. The fragments are separated by liquid chromatography with an acetonitrile gradient and analysed in mass spectrometry, by the following procedure:

A 100 μg of fraction purified by chromatofocusing or by CEX were freeze-dried and re-dissolved in 20 μl of a digestion buffer (50 mM NaH₂PO₂ and 150 mM NaCl, pH 6.30), and 100 IU of IdeS enzyme were added by following the instruction of the enzyme kit (FabRICATOR Kit, Genovis, Lund, Sweden). The preparation was incubated at 37° C. for 1 hour with microwave assistance at a power of 50 W (CEM Discover System, CEM, Matthews, NC, USA) for improving hydrolysis. Next, 25 μl of a denaturing buffer (8M urea and 0.4M of NH₄HCO₃, pH 8.0) were added, followed by 5 μl of a dithiothreitol (DTT) solution at 250 mM. The sample was incubated at 50° C. for 20 minutes with microwave assistance at a power of 50 W for ensuring complete reduction of the protein, which was then analysed by liquid chromatography—mass spectrometry (LC-MS).

An aliquot of the reaction mixture corresponding to an amount of 20 μg was injected on a reverse phase ProSphere C4 column (150×2.1 mM, 5 μm, Alltech) equilibrated to 70° C. at a flow of 350 p1/min. The reverse phase chromatography was carried out by using an ultra-performing liquid chromatography system (UPLC,

Acquity UPLC, Waters, Milford, MA, USA). The gradient was generated by using trifluoroacetic acid (TFA) at 0.1% as a mobile phase A and acetonitrile comprising 0.1% of TFA as a mobile phase B. After isocratic elution at 10% of B for 5 minutes, B was increased to 27% for 5 minutes and then to 40% for a further 10 minutes. The column was then washed for 3 minutes with 90% of B and re-equilibrated for 2 minutes at 10% of B, giving an overall duration of 25 minutes.

The eluted species were then analysed with a mass spectrometer QSTAR (QSTAR XL, Applied Biosystems, Toronto, Canada) operating in a positive ion mode of 500 to 3,000 m/z and calibrated according to the procedure described by the manufacturer for renin.

### RESULTS

### Separation of the Charge Isoforms by Chromatofocusing

The chromatograms of the 3 separations are shown in FIG. 1, which shows that they may be perfectly superposed, thus demonstrating the reproducibility of the separation method by chromatofocusing. Because of the use of an anion exchange resin and of a decreasing pH gradient, the basic isoforms are eluted first, followed by the major isoform, and then by the acidic isoforms.

The fractions 33 to 50 were collected for subsequent analysis of their biochemical and effector properties.

### Separation of the Charge Isoforms by Cation Exchange Chromatography (CEX)

The chromatograms of 11 separations by cation exchange chromatography (CEX) of the charge isoforms are shown in FIG. 2. Because of the use of a cation exchange resin, the acidic isoforms are eluted first, followed by the major isoform, and then by the basic isoforms.

Peak 4 (P4, main peak) was reanalysed in CEX in order to check the efficiency of the purification. The percentages of acidic, main and basic isoforms obtained before and after separation with CEX are shown in FIG. 3 and in Table 4 below, and clearly show the efficiency of purification of the main peak.

TABLE 4
Percentages of acidic, main and basic isoforms obtained
before and after separation with CEX

| Sample | Acidic forms | Main peak | Basic forms |
|---|---|---|---|
| Before separation | 12.6% | 58.7% | 28.7% |
| After separation | 3.5% | 93.4% | 3.1% |

### Analysis of the Binding to the CD16 Receptor by BIACORE

The capability of the various fractions separated by cation exchange chromatography, comprising different charge isoforms, of binding to the receptor CD16 was tested.

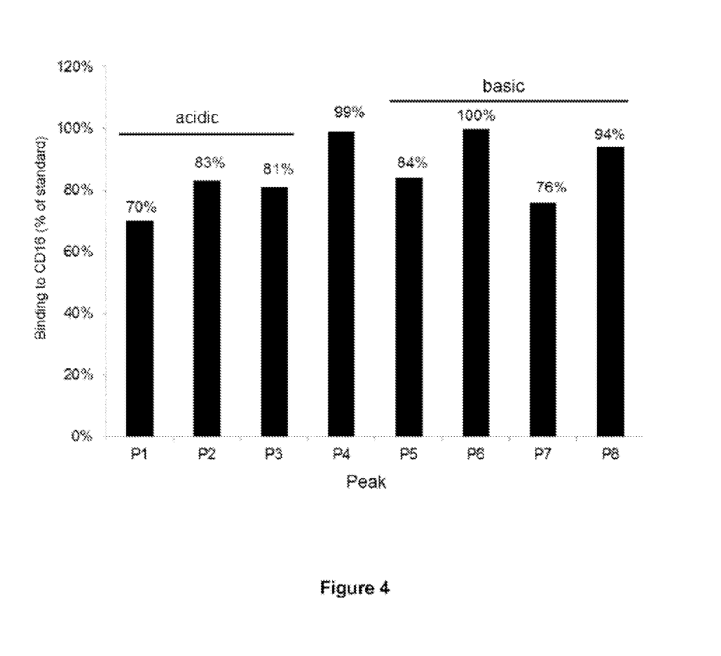

The results are shown in FIG. 4, and show a loss of affinity for the acidic forms (P1 to P3) and for the peak 7 (P7), but not for the other basic forms (P6, P8).

### Activation of Effector Cells via CD16 (CD16 Jurkat Test)

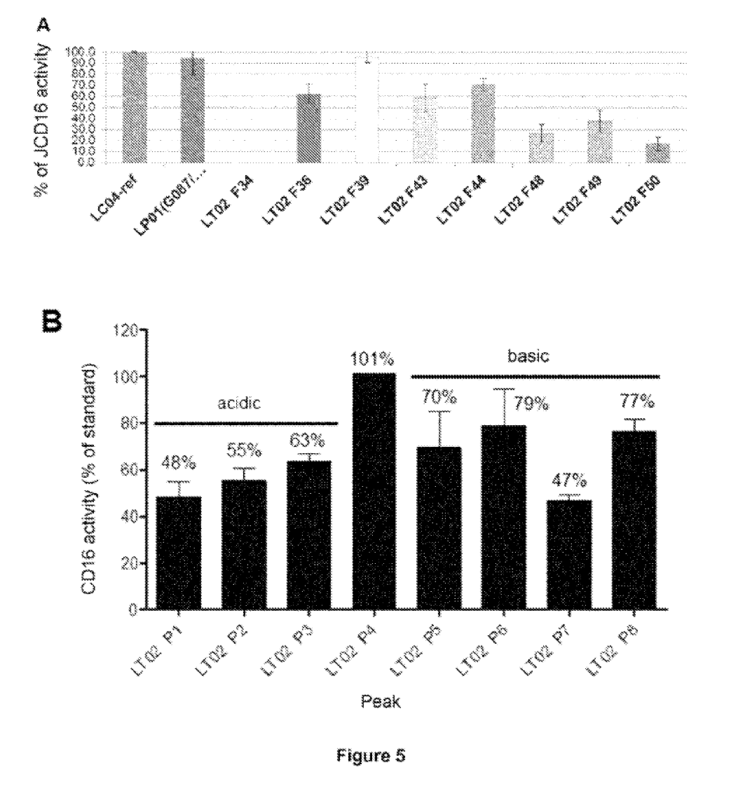

The capability of the various fractions separated by chromatofocusing and by cation exchange chromatography (CEX), comprising different charge isoforms, of inducing a response of effector cells via the CD16 receptor (FcγRIII) was tested. The results are shown in FIG. 5 (CEX), and Table 5 (separation by chromatofocusing) below.

In each case, it is observed that the fraction corresponding to the major isoform induces activation of the CD16 Jurkat cells which is significantly more substantial than that of the fractions comprising the acidic or basic isoforms.

Thus, the capability of the various charge isoforms of activating effector cells via CD16 varies significantly, the major isoform having a significantly improved capability as compared with the other isoforms of activating effector cells expressing CD16.

The test described above, which measures the amount of secreted IL-2 by Jurkat cells transfected with the receptor CD16 in the presence of an antibody composition, was shown to be representative of the capability of this antibody composition of inducing ADCC by the effector cells expressing CD16 (WO2004/024768). Therefore, the results shown in Tables 4 to 6 below indicate that the purified fractions corresponding to the major peak of chromatofocusing or of CEX before purification has a significantly improved capability as compared with the other isoforms and as compared with a total composition comprising all the isoforms for inducing ADCC via the effector cells expressing CD16.

TABLE 5
Activation of the CD16 Jurkat cells by a reference composition,
by the total composition before separation, and by the fractions
F36 (basic isoforms), F39 (major isoform), and F43, F44, F48,
F49 and F50 (increasingly acid isoforms) from separation by
chromatofocusing.

| | CD16 Jurkat activity |
|---|---|
| Sample | (% of the reference composition) |
| reference composition | 100% |
| total composition before separation | 80% |
| F36 (basic isoform) | 62% |
| F39 (major isoform) | 96% |
| F43 (acidic isoform) | 59% |
| F44 (acidic isoform) | 71% |
| F48 (acidic isoform) | 27% |
| F49 (acidic isoform) | 38% |
| F50 (acidic isoform) | 17% |

### Complement-Dependent Cytotoxicity (CDC)

The capability of the various fractions separated by cation exchange chromatography (CEX), comprising different charge isoforms, of inducing a complement-dependent cytotoxic response (CDC) was measured.

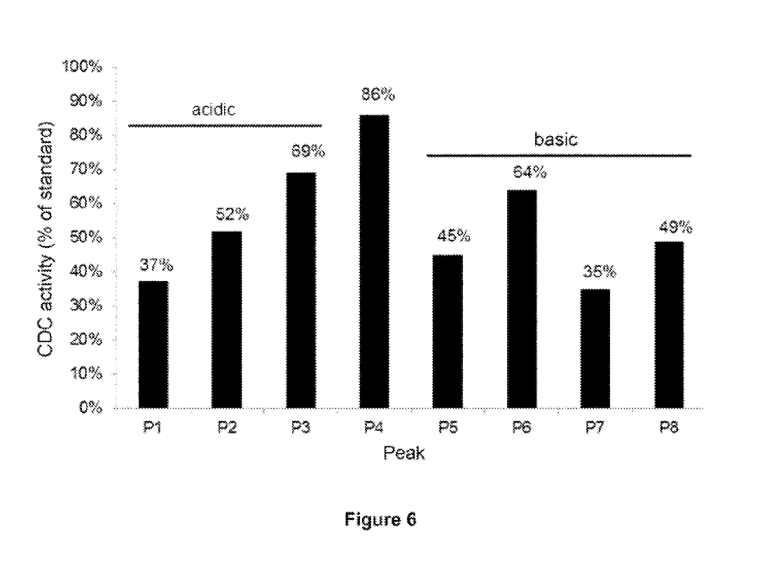

The results are shown in FIG. 6, and show a strong loss of activity for the acidic forms (P1: 37%, P2: 52%, and P3: 69%) and the basic forms (P5: 45%, P6=K1: 64%, P7: 35%, and P8=K2: 49%) as compared with the main peak (P4). The peak corresponding to the major isoform (P4) therefore induces a significantly greater CDC response than the fractions comprising acidic or basic isoforms.

### Characterisation of the Isoforms by Mass Spectrometry

The fractions purified by chromatofocusing and the fractions purified by CEX were analysed by LC-MS in order to characterise the percentage of heavy chains with or without an N-terminal lysine.

For the fractions purified by chromatofocusing and the fractions purified by CEX corresponding to the major peak before separation, the analysis showed that more than 95% of the heavy chains do not comprise any C-terminal lysine.

### CONCLUSION

The results shown above show that the charge isoforms of an antibody composition corresponding to the major peak of a separation by ion exchange chromatography (CEX) or by chromatofocusing have a significantly larger capability than the acidic or basic isoforms of the same antibody composition of activating the effector cells via the receptor FcγRIII (CD16), and also via the complement. The use of purified fractions corresponding to this major peak therefore would allow a further increase in the effector properties via CD16 (ADCC, secretion of cytokines) within the scope of pathologies treated by monoclonal antibodies in which ADCC or the CDC response play an important role, such as notably the preventing of allo-immunization, or the treatment of cancers, of infectious diseases, and of auto-immune diseases.

### BIBLIOGRAPHIC REFERENCES

Ahmad et al. FASEB J. 1996. 10:258-266.
Albrecht M T, et al. INFECTION AND IMMUNITY, Nov. 2007, p. 5425-5433.
Almagro et al. Frontiers in Bioscience 13, 1619-1633, Jan. 1, 2008.
Antes B, et al. J Chromatogr B Analyt Technol Biomed Life Sci. 2007 Jun 1;852(1-2):250-6.
Beliard Ret al. Br J Haematol. 2008 Apr;141(1):109-19.
Bruggermann et al., Year in Immuno., 7:33 (1993);
Cardarelli et al. Clin Cancer Res 2009 April 28;15:3376-3383,
Cardarelli et al. Cancer Immunol Immunother. 2010. 59. 257-265,
Cartron G, et al. Blood. 2002 Feb. 1;99(3):754-8.
Casadevall A. et al. Emerg Infect Dis. 2002 Aug;8(8):833-41;
Chan AC, et al. Nat Rev Immunol. 2010 May;10(5):301-16.
Chevreux G et al. Anal Biochem. 2011 Aug 15;415(2):212-4;
Cibelli et al., 1998 Science, 280: 1256-1258
Dall'Acqua et al. 2002, J Immunol.;169:5171 -80.
Dall'Acqua et al. 2006, J. Biol. Chem.;281:23514-24. (a)
Dall'Acqua et al. J Immunol 2006; 177:1129-1138. (b)
Duchosal et al. Nature 355:258 (1992)
Edelman, G. M. et al., Proc. Natl. Acad. USA, 63, 78-85 (1969).
Edwards J C, et al. Nat Rev Immunol. 2006 May;6(5):394-403.
EP 1 176 195 A1,
EP 1 308 456,
EP 1 829 961,
Farnan D, Moreno G T. Anal Chem. 2009 Nov 1;81(21):8846-57.
Fisher R, et al. Vaccine 21 (2003) 820-825.
Forthal et al., J Immunol 2010;185;6876-6882,
Gandhi S, et al. Pharm Res. 2012 Jan;29(1):209-24.
Gene Targeting, A Practical Approach, IRL Press at Oxford University Press (1993)
Gordon et al., 1980 Proc Natl Acad Sci U S A.;77:7380-4
Herbst R. et al. J Pharmacol Exp Ther. 2010 October;335(1):213-22,
Hinton et al. 2004, J Biol Chem.;279:6213-6.
Hoogenboom et al., J. Mol. Biol., 227:381 (1991);
Ianello A, et al. Cancer and Metastasis Reviews 24: 487-499, 2005.
Idusogie E E et al. J Immunol. 2001; 166:2571-5.
Imai-Nishiya et al., BMC Biotechnology 2007, 7:84;
Jakobovits et al., Proc. Natl. Acad. Sci. USA. 90:2551 (1993) (a);
Jakobovits et al., Nature, 362:255-258 (1993) (b);
Jones et al. Nature, 321: 522-525, 1986;
Kabat et al., Sequences of Proteins of Immunological Interest, 5th Ed. Public Health Service, National Institutes of Health, Bethesda, Md. (1991).
Kanda Y et al., Journal of Biotechnology 130 (2007) 300-310,
Khawli L A, et al. MAbs. 2010 November-December;2(6):613-24.
Lazar, G. A., et al. Proc Natl Acad Sci U S A. 103(11): 4005-10.
Ma J K, et al. Nat Rev Genet. 2003 October;4(10):794-805.
Manipulating the Mouse Embryo, A Laboratory Manual, Second edition, Cold Spring Harbor Laboratory Press (1994);
Marks et al., J. Mol. Biol., 222:581-5 597 (1991);
McAtee C P et al. Curr Protoc Protein Sci. 2012 August;Chapter 8:Unit 8.10,
Miao C, et al. Journal of General Virology (2009), 90, 1119-1123.
Moore G L. et al. mAbs 2:2, 181-189; March/April, 2010;
Mori K, et al. Biotechnol Bioeng. 2004 Dec. 30;88(7):901-8,
Olivier S. et al. MAbs. 2010 July-August; 2(4): 405-415,
Presta L G. Adv Drug Deliv Rev. 2006 Aug. 7;58(5-6):640-56.
Rea J C, et al. J Pharm Biomed Anal. 2011 Jan. 25;54(2):317-23.
Rea Jennifer C. Innovations in Biotechnology. InTech. Feb. 17, 2012. Chapter 19.
Riechmann et al. Nature, 332: 323-327, 1988,
Ryan et al., 1997 Science; 278: 873 -876;

Satoh M, et al. Expert Opin Biol Ther. 2006 November;6(11):1161-73.
Schillberg S, et al. Vaccine 23 (2005) 1764-1769.
Shields R L, et al. J Biol Chem. 2001 Mar. 2;276(9):6591-604.
Stoger E, et al. Molecular Breeding 9: 149-158, 2002.
Suzuki et al. Clin Cancer Res 2007 Mar. 15;13:1875-1882,
Umana et al. Nat Biotechnol. 1999 February;17(2):176-80,
U.S. Pat. No. 5,591,669,
U.S. Pat. No. 5,598,369,
U.S. Pat. No. 5,545,806,
U.S. Pat. No. 5,545,807,
U.S. Pat. No. 6,150,584
Vaughan et al. Nature Biotech 14:309 (1996)
Velders MP et al., Br J Cancer. 1998 Aug.;78(4):478-83.
Verhoeyn et al. BioEssays, 8: 74, 1988,
Verhoeyen et al. Science, 239: 1534-1536, 1988;
Vlasak J, Ionescu R. Curr Pharm Biotechnol. 2008 Dec.;9(6):468-81.
Wallace P K et al., J Leukoc Biol. 1994 June;55(6):816-26.
Weiner L M, et al. Nat Rev Immunol. 2010 May;10(5):317-27.
WO9004036A1,
WO9517085A1,
WO9951642,
WO0026357A2,
WO00/42072,
WO01/77181,
W001 /26455A1,
WO02/060919A2,
WO2004/024768,
WO2004/024866,
WO2004/029207,
WO2004/050847A2,
WO2004/063351
WO2004/074455
WO2005/033281 A2,
WO2007/048077A2
WO2007/106078A2,
WO2010/045193,
WO2010/106180A2,
WO2011/009623,
WO2012/041768A1
Wright A, Morrison SL. J Exp Med. 1994 Sep. 1;180(3):1087-96,
Yamane-Ohnuki N. et al. Biotechnol Bioeng. 2004 Sep. 5;87(5):614-22,
Zeitlin L. et al. Microbes Infect. 2000 May;2(6):701-8,
Zhang T. et al. J Chromatogr A. 2011 Aug. 5;1218(31):5079-86.

1. A monoclonal antibody composition which may be obtained by a method comprising:
a) producing a monoclonal antibody composition from a cell clone, a non-human transgenic animal or a transgenic plant,
b) fractionating the composition obtained in step a) by chromatography, and
c) combining one or several chromatographic fractions obtained in step b), corresponding to the major peak of the chromatogram, the thereby obtained monoclonal antibody composition being enriched in said major peak, the latter representing at least 85% of the chromatogram of the composition obtained in step c),
for its use as a medicament.

2. The monoclonal antibody composition according to claim 1, for its use as a medicament according to claim 1, characterized in that the fractionation of step b) is achieved by ion exchange chromatography, by chromatofocusing or by hydrophobic interactions chromatography.

3. The monoclonal antibody composition according to claim 2, for its use as a medicament according to claim 2, characterized in that ion exchange chromatography uses one of the following elution means:
ionic force gradient; and/or
pH gradient; or
a displacement molecule.

4. The monoclonal antibody composition according to any of claims 1 to 3, for its use as a medicament according to any one of claims 1 to 3, characterized in that at least 95% of the heavy chains of the antibodies present in the composition do not comprise any C-terminal lysine residue.

5. A monoclonal antibody composition, wherein at least a 95% of the heavy chains of the antibodies present in the composition do not comprise any C-terminal lysine residue, for its use as a medicament.

6. The monoclonal antibody composition according to any one of claims 1 to 5, for its use as a medicament according to any one of claims 1 to 5, characterized in that the antibody is directed against a non-ubiquitous antigen present on healthy donor cells, an antigen of a cancer cell, an antigen of a cell infected by a pathogenic agent, or an antigen of an immune cell.

7. The monoclonal antibody composition according to any one of claims 1 to 6 for its use as a medicament according to any one of claims 1 to 6, characterized in that the antibody is an anti-Rhesus D antibody and the composition is intended for preventing allo-immunization in Rhesus-negative individuals.

8. The monoclonal antibody composition according to any one of claims 1 to 6 for its use as a medicament according to any one of claims 1 to 6, characterized in that the antibody is directed against an antigen of a cancer cell and the composition is intended for treating a cancer.

9. The monoclonal antibody composition according to any one of claims 1 to 6 for its use as a medicament according to any one of claims 1 to 6, characterized in that the antibody is directed against an antigen of a cell infected by a pathogenic agent and the composition is intended for treating an infection by said pathogenic agent.

10. The monoclonal antibody composition according to any one of claims 1 to 6 for its use as a medicament according to any one of claims 1 to 6, characterized in that the antibody is directed against an antigen of an immune cell and the composition is intended for treating an auto-immune disease.

11. The monoclonal antibody composition according to any one of claims 1 to 10 for its use as a medicament according to any one of claims 1 to 10, characterized in that the antibody comprises a modification of the Fc fragment enhancing its binding to the FcγRIII receptor and its effector properties via the FcγRIII receptor.

12. The monoclonal antibody composition according to claim 11 for its use as a medicament according to claim 11, characterized in that the antibody comprises at least one mutation at certain amino-acid residues of the Fc fragment.

13. The monoclonal antibody composition according to claim 11 or claim 12 for its use as a medicament according to claim 11 or claim 12, characterized in that it comprises a fucose content of less than or equal to 65%.

14. The monoclonal antibody composition according to any one of claims 1 to 10 for its use as a medicament according to any one of claims 1 to 10, characterized in that the antibody comprises a modification of the Fc fragment enhancing its binding to the protein C1q and its effector properties via the complement.

15. Use of a chromatography fractionation step for increasing the ability of a monoclonal antibody composition directed against a given antibody to induce antibody-dependent cell cytotoxicity (ADCC) of target cells expressing said antigen by the effector cells of the immune system expressing the FcγRIII (CD16) receptor.

16. Use of a chromatography fractionation step for increasing the ability of a monoclonal antibody composition directed against a given antibody to induce complement-dependent cytotoxicity (CDC) of target cells expressing said antigen by the complement.
